(19) United States
(12) Patent Application Publication
(10) Pub. No.: US 2022/0039739 A1
(43) Pub. Date: Feb. 10, 2022

(54) METHODS FOR EVALUATING PATIENTS

(71) Applicant: Biogen MA Inc., Cambridge, MA (US)

(72) Inventors: Donald R. Johns, Woburn, MA (US); Dawei Liu, Sharon, MA (US); Toby Ferguson, Concord, MA (US); Jesse M. Cedarbaum, Woodbridge, CT (US)

(21) Appl. No.: 17/206,785

(22) Filed: Mar. 19, 2021

Related U.S. Application Data
(63) Continuation of application No. 15/629,424, filed on Jun. 21, 2017, now abandoned.
(60) Provisional application No. 62/352,832, filed on Jun. 21, 2016.

(51) Int. Cl. A61B 5/00; A61B 5/11; A61B 5/145; A61B 5/22; A61B 5/24; A61B 5/389

(52) U.S. Cl. CPC A61B 5/4519; A61B 5/389; A61B 5/1121; A61B 5/4842; A61B 5/4571; A61B 5/7246; A61B 5/4585; A61B 5/4595; A61B 5/4576; A61B 5/458; A61B 5/459; A61B 5/4848; A61B 5/14546; A61B 5/224; A61B 5/14542; A61B 5/24; A61B 5/1123

(57) ABSTRACT

Methods for evaluating subjects having conditions associated with loss of muscle function (e.g., a motor neuron disease, a neuromuscular disease, or a myopathy) by measuring muscle function (e.g., muscle strength) are disclosed.

### RELATED APPLICATIONS

This application is a Continuation of U.S. Ser. No. 15/629,424, filed Jun. 21, 2017, which claims priority to U.S. Ser. No. 62/352,832 filed Jun. 21, 2016, the contents of which are hereby incorporated by reference in their entirety.

### FIELD OF THE INVENTION

The present invention relates generally to methods and devices for measuring muscle function in patients having, or at risk of having, a disease or disorder associated with neurological and/or muscular degeneration for predictive, diagnostic, prognostic, or evaluative purposes.

### BACKGROUND

Amyotrophic lateral sclerosis (ALS) is a nerve and muscle disease that is characterized by progressive loss of motor neurons and therefore muscle function. Traditional methods for tracking disease progression in ALS clinical studies utilize survival and the Amyotrophic Lateral Sclerosis Functional Rating Scale (ALSFRS) as the endpoints. The ALSFRS is a validated questionnaire-based scale that rates on a scale of 0-4 physical function in carrying out activities of daily living (ADL) of patients with ALS (Brooks B R, et al., Arch Neurol 1996; 53:1441-7 and Cedarbaum J M, et al., J Nerol Sci 1997; 152 (Suppl):1-9). A revised ALSFRS (ALSFRS-R) incorporates assessment of respiratory function and has been shown to be more sensitive and has better ability to predict survival than the original ALSFRS (Cedarbaum et al., J Neurol Sci 1999; 13-21). However, the limitations of the traditional endpoints of survival and the ALSFRS (or ALSFRS-R) include: a very long follow-up time (e.g., 12-18 months, a very large sample size (e.g., typically requires 300-400 participants in each arm in a phase 3 clinical trial), and low sensitivity to tracking disease progression within a short time follow-up.

Thus, there exists a need for methods that are more sensitive and specific for tracking disease progression, evaluating patients, and identifying therapeutics with clinical efficacy for diseases associated with loss of muscle function, such as ALS. Specifically, there exists a need for methods that detect changes in disease progression in a shorter time period (e.g., less than 18 months), detect disease at an early stage, require a smaller number of participants, and provide clinically meaningful data.

As loss of muscle strength occurs in many different diseases that are associated with loss of muscle function or muscle strength, the measurement of muscle function or strength should be the most sensitive endpoint for tracking disease progression, assessing disease severity, identifying therapeutics that could delay or ameliorate symptoms, and monitoring patients. Current methods for using muscle strength to track disease progression include aggregated values based on strength measures from multiple muscles, for example, megascore, which is the average of z-scores for each individual muscles, and average percentage of muscle strength normalized based on patient-level characteristics such as age, gender and weight. These endpoints track disease progression well. In at least the past 20 years in ALS research and clinical studies, no study has been able to show that they are more sensitive than ALSFRS-Rand thus they are rarely used as a primary endpoint in clinical studies in ALS.

### SUMMARY OF THE INVENTION

The present disclosure features, at least in part, a novel method for evaluating subjects having a disease or disorder associated with loss of muscle function (e.g., a motor neuron disease, a neuromuscular disease, or a myopathy) by measuring muscle function (e.g., muscle strength) and determining when a muscle or a preselected combination of muscles reaches zero strength or near-zero function. Methods described herein incorporate measures of muscle strength which are shown to be more sensitive to track disease progression and evaluate patients. Methods described herein focus on a landmark event in disease progression based on strength measures, which is the time to zero strength (e.g., the muscle has lost all or substantially all function). In earlier studies, the assessment of muscles that have lost all or substantially all function in the muscles was not treated specifically from other non-zero strength measures and mostly considered not to contain any information useful for evaluating patients or tracking disease progression. The present disclosure demonstrates that the utilization of a Zero Function Value, e.g., when the muscle has lost all or substantially all function, allows a more accurate and sensitive method for evaluating and tracking a disease associated with loss of muscle function as described herein.

The methods described herein provide one or more of the following advantages over traditional endpoints and methods of evaluation (e.g., the ALSFRS-R): i) a muscle-function based test for diseases that are characterized by a loss of function; ii) a very sensitive method to track disease progression; iii) a very sensitive method to track early stage disease; iv) a method that is not confounded by variables such as age, gender, or body weight; v) ease of measurement; vi) shorter time period required for detecting changes in disease progression or therapeutic efficacy; and vii) smaller number of participants required for clinically meaningful results.

Accordingly, in one aspect, the invention features, a method of evaluating a subject having a disease associated with loss of muscle function, e.g., a motor neuron disease or related neuromuscular disorder, e.g., ALS (e.g., bulbar or non-bulbar ALS) or SMA. The method includes:

providing, e.g., by measuring, a value for function, e.g., Muscle Function or for a Zero Muscle Function Factor (ZMF Factor) for a Sentinel Muscle of the subject, thereby evaluating the subject having a disease associated with loss of muscle function.

In an embodiment, the method further comprises:

providing, e.g., by determining, if a value for ZMF Factor or Muscle Function, is a Zero Function Value.

In an embodiment, the method further comprises:

providing, e.g., by determining, the time elapsed between a preselected time point or event, e.g., time from onset of disease, time from diagnosis, or time from onset of muscle weakness, and the time at which Zero Function Value is reached (the time to Zero Function Value, or T_{ZFV}), for the Sentinel Muscle.

In another aspect, the invention features, a method of evaluating a subject having a disease associated with loss of muscle function, e.g., a motor neuron disease or related neuromuscular disorder, e.g., ALS (e.g., bulbar or non-bulbar ALS) or SMA. The method includes:

a) providing, e.g., by measuring, a value for Muscle Function or for a Zero Muscle Function Factor (ZMF Factor) for a muscle of the subject;

b) optionally, determining if the value for ZMF Factor or Muscle Function, is a Zero Function Value; and

c) determining the time elapsed between a preselected time point or event, e.g., time from onset of disease, time from diagnosis, or time from onset of muscle weakness, e.g., Sentinel Muscle weakness, e.g., anterior tibialis weakness, and the time at which Zero Function Value is reached (the time to Zero Function Value, or T_{ZFV}), thereby evaluating the subject having a disease associated with loss of muscle function.

In an embodiment the method includes steps a) and c).

In an embodiment the method includes steps a), b) and c).

In an embodiment, the muscle is a Sentinel Muscle.

In another aspect, the invention features a method of evaluating a subject having a motor neuron disease or related neuromuscular disorder. The method includes:

a) providing, e.g., by measuring, a value for Muscle Function or for a Zero Muscle Function Factor (ZMF Factor) for a muscle of the subject;

b) optionally, comparing the provided value for Muscle Function or ZMF Factor to a Zero Function Value to determine if the muscle has reached or has not reached zero-function; and

c) determining the T_{ZFV},

thereby, responsive to the ZMF Factor, Muscle Function, or T_{ZFV}, evaluating the subject.

In certain embodiments, the method further comprising, responsive to the value for ZMF Factor, Muscle Function, or T_{ZFV}, classifying the subject.

In certain embodiments, the method further comprises, responsive to the value for T_{ZFV}, classifying the subject.

In another aspect, the invention features a method of evaluating or treating a subject having a disease associated with loss of muscle function, e.g., a motor neuron disease or related neuromuscular disorder, e.g., ALS or SMA, comprising:

acquiring, e.g., by determining, or by receiving from another entity, a value from an evaluation made by the methods described herein;

thereby evaluating or treating the subject.

In certain embodiments, the method comprises:

a) responsive to the value, selecting a treatment for the subject; or

b) acquiring from another entity, a selection of treatment for the subject, the selection having been made responsive to the value.

In some embodiments, the method comprises administering the treatment to the subject.

In some embodiments, the method further comprises comparing the value for ZMF Factor, Muscle Function or T_{ZFV}, with a reference value, e.g., to determine if the muscle has reached Zero Function.

In some embodiments, the method further comprises comparing the value for T_{ZFV}, with a reference value.

In some embodiments, e.g., for a comparison of a value for ZMF Factor, or Muscle Function, a reference value is a previous value for a subject, e.g., a value determined at onset of disease, at diagnosis, or at onset of muscle weakness, e.g., anterior tibialis weakness. In some embodiments, the reference value is 90%, 91%, 92%, 93%, 94%, 95%, 96%, 97%, 98%, or 99% of a value determined at onset of disease, at diagnosis, or at onset of muscle weakness, e.g., Sentinel Muscle weakness, e.g., anterior tibialis weakness.

In some embodiments, e.g., for a comparison of a value for T_{ZFV}, a reference value can be a value of T_{ZFV} of another subject or an average from a plurality of subjects.

In some embodiments, the method comprises providing a value for Muscle Function or for a Zero Muscle Function Factor (ZMF Factor) once, twice, three times, four times, five times, six times, seven times, eight times, nine times, or ten times. In some embodiments, the method comprises providing a value for Muscle Function or for a Zero Muscle Function Factor (ZMF Factor) once a month for at least, 1, 2, 3, 4, 5, 6, 12, 18 or 24 months.

In some embodiments, providing comprises performing a measurement. In certain embodiments, providing comprises receiving a value from another party.

In some embodiments, the disease associated with loss of muscle function is a motor neuron disease, neuromuscular disorder, or a myopathy. In certain embodiments, the disease associated with loss of muscle function is one or more selected from: amyotrophic lateral sclerosis (ALS) (e.g., bulbar or non-bulbar ALS), spinal muscular atrophy (SMA), spinobulbar muscular atrophy (SBMA), polymyositis, inclusion body myositis, a motor neuropathy, and distal hereditary motor neuropathy. In some embodiments, the disease associated with loss of muscle function is ALS, e.g., bulbar or non-bulbar ALS. In other embodiments, the disease associated with loss of muscle function is SMA.

### Uses

In some embodiments, muscle function is evaluated by or expressed as one or more of:
a) the ability to exert force, e.g., force exerted by a limb or extremity, e.g., a distal limb or extremity (e.g., this can be evaluated upon one effort, or upon repeated efforts, e.g., upon repeated efforts within a preselected time period);
b) peak force, e.g., peak force exerted by a limb or extremity, e.g., a distal limb or extremity (e.g., this can be evaluated upon one effort, or upon repeated efforts, e.g., upon repeated efforts within a preselected time period);
c) the ability to maintain force, e.g., ability to maintain force exerted by a limb or extremity, e.g., ability to repeat a force level or repeat a motion by a limb or extremity, e.g., for a preselected time (e.g., this can be evaluated upon one effort, or upon repeated efforts, e.g., upon repeated efforts within a preselected time period);
d) an electrophysiological parameter, e.g., neuron conductivity, neuromuscular (e.g., axon to muscle cell) junction transmission, ability of an axon to elicit a contraction of a muscle (e.g., this can be evaluated upon once, or upon repeatedly, e.g., repeated measurements within a preselected time period), motor unit number estimation (MUNE), an electrical impedance myography (EIM) characteristic (e.g., voltage, current, phase, frequency dependence, anisotropy), or axonal excitability;
e) the ability to assume an anti-gravity position, e.g., the ability to assume an anti-gravity position by a limb or extremity, e.g., a distal limb or extremity, e.g., for a preselected time period (e.g., this can be evaluated upon one effort, or upon repeated efforts, e.g., upon repeated efforts within a preselected time period);
f) range of motion, e.g., the range of motion demonstrated by a limb or extremity, e.g., a distal limb or extremity (e.g., this can be evaluated upon one effort, or upon repeated efforts, e.g., upon repeated efforts within a preselected time period);
g) speed attainable, e.g., speed attainable of a limb or extremity, e.g., a distal limb or extremity, e.g., with regard to a preselected motion (e.g., this can be evaluated upon one effort, or upon repeated efforts, e.g., upon repeated efforts within a preselected time period); or
h) acceleration e.g., ability of the muscle to accelerate (e.g., this can be evaluated upon one effort, or upon repeated efforts, e.g., upon repeated efforts within a preselected time period).

In certain embodiments, evaluating the subject comprises one or more of:
evaluating disease progression;
evaluating for Zero Function Value for a muscle;
determining if a muscle in a subject has reached Zero Function;
determining prognosis;
predicting the outcome of disease, e.g., ALS, e.g., bulbar or non-bulbar ALS, in the subject;
predicting the survival of the subject;
evaluating the effectiveness of a treatment;
determining a preferred treatment regimen;
classifying the subject as having reached Zero Function for a muscle;
classifying the subject as having not reached Zero Function for a muscle;
comparing the Muscle Function, ZMF Factor, or T_{ZFV} slope to the ALSFRS-R slope;
comparing the Muscle Function, ZMF Factor, or T_{ZFV} slope to the survival slope; or
identifying a pattern, e.g., an unusual pattern, of disease progression;

In certain embodiments evaluating comprises one or more of:
comparing the T_{ZFV} slope to the ALSFRS-R slope; or
comparing the T_{ZFV} slope to the survival slope.

In some embodiments, evaluating the subject comprises, responsive to the value for Muscle Function, ZMF Factor, or T_{ZFV}, classifying, selecting, modifying prognosis or treatment or making a prediction about, the subject. In certain embodiments, evaluating, classifying, selecting, modifying prognosis or treatment or making a prediction about, the subject comprises:
predicting the outcome of disease, e.g., ALS, e.g., bulbar or non-bulbar ALS, in the subject;
predicting the survival of the subject;
evaluating the effectiveness of a treatment;
determining a preferred treatment regimen;
classifying the subject as having reached Zero Function for a muscle;
classifying the subject as having not reached Zero Function for a muscle;
comparing a parameter related to Muscle Function, ZMF Factor, or T_{ZFV}, e.g., Muscle Function, ZMF Factor, or T_{ZFV}, slope, to another parameter;
comparing a parameter related to Muscle Function, ZMF Factor, or T_{ZFV}, e.g., Muscle Function, ZMF Factor, or T_{ZFV}, slope, to a parameter related to ALSFS-R, e.g., the ALSFRS-R slope;
comparing Muscle Function, ZMF Factor, or T_{ZFV}, slope to the survival slope; or
identifying unusual patterns of disease progression.

In some embodiments, the method comprises, responsive to the evaluation, selecting a course of action for the subject, e.g., selecting or modifying a treatment. In certain embodiments, the method comprises, responsive to the evaluation, implementing a course of action for the subject, e.g., administering a treatment to the subject. In some embodiments, the method comprises continuing a preexisting treatment. In certain embodiments, the method comprises continuing a preexisting treatment, under a new regimen, e.g., lower or higher dosage, or more or less frequent administrations. In some embodiments, the method comprises continuing a preexisting treatment, and initiating a second treatment. In certain embodiments, the method comprises discontinuing a preexisting treatment, and initiating a second treatment.

In some embodiments, the method comprises, responsive to the evaluation, classifying or selecting the subject. In certain embodiments, the predicting or classifying is performed within a preselected time of:
initial diagnosis of the disease associated with muscle function loss, e.g., ALS, e.g., bulbar or non-bulbar ALS;
initial administration of a treatment for the disease associated with muscle function loss, e.g., ALS, e.g., bulbar or non-bulbar ALS;
initiation into clinical trial;
completion of a clinical trial;
modification of treatment for the disease associated with muscle function loss, e.g., ALS, e.g., bulbar or non-bulbar ALS;
abandonment or conclusion of a treatment for the disease associated with muscle function loss, e.g., ALS, e.g., bulbar or non-bulbar ALS;
failure to respond to a treatment for the disease associated with muscle function loss, e.g., ALS, e.g., bulbar or non-bulbar ALS;
presentation with a preselected symptom, or disease stage;
prior to presentation of a preselected symptom.

In some embodiments, the preselected time is 1 day, 10 days, 20 days, 30 days, 60 days, 90 days, 120 days, 150 days, 180 days, 210 days, 240 days, 270 days, 300 days, 330 days, 365 days, 14 months, 16 months, 18 months, 20 months, 22 months, 24 months, 30 months, or 36 months. In certain embodiments, the preselected time is less than 48 months, less than 42 months, less than 36 months, less than 30 months, less than 24 months, less than 16 months, or less than 12 months.

In some embodiments, the evaluation shows Zero Function within the preselected time. In certain embodiments, responsive to the determination that the subject has not reached Zero Function, classifying the subject as being in need of subsequent evaluation.

In certain embodiments, classifying comprises:

responsive to a value for Muscle Function or ZMF Factor based determination that the subject has reached Zero Function, assigning a first classification to the subject, e.g., classifying the subject as having Zero Function; or

responsive to a value for Muscle Function or ZMF Factor based determination that the subject has not reached Zero Function, assigning a second classification to the subject, e.g., classifying the subject as being in need of a subsequent evaluation for Zero Function.

In some embodiments the method further comprises repeating one or more or all of:

a) providing, e.g., by measuring, a value for Muscle Function or for a Zero Muscle Function Factor (ZMF Factor) for a muscle of the subject;

b) determining if the value for ZMF Factor or Muscle Function, is a Zero Function Value; and

c) determining the T_{ZFV}.

In certain embodiments, the method further comprises repeating steps a), b), and c) until the subject has reached zero function. In some embodiments, the method further comprises repeating steps a), b), and c) until a value for or ZMF Factor or Muscle Function that indicates Zero Function is obtained. In further embodiments, the subject has previously been classified as not having reached Zero Function.

In some embodiments, the method comprises receiving a value for Muscle Function or ZMF Factor from a prior evaluation.

### Muscle Groups

In some embodiments, the muscle is a Sentinel Muscle (e.g., FDIO or ANKLDOR). In certain embodiments, the muscle is selected from:
the left first dorsal interosseous (L-FDIO);
the right first dorsal interosseous (R-FDIO);
the left ankle dorsiflexion (L-ANKLDOR); or
the right ankle dorsiflexion (R-ANKLDOR).

In some embodiments, the method comprises evaluating:
one of:
i) the left first dorsal interosseous (L-FDIO); and
ii) the right first dorsal interosseous (R-FDIO); and
one of:
iii) the left ankle dorsiflexion (L-ANKLDOR); and
iv) the right ankle dorsiflexion (R-ANKLDOR).

In the instant application, including in the Figures, the location of the muscle on left or right is indicated by an L or R in front of the muscle label or at the end of the muscle label.

In some embodiments, the method comprises evaluating: i) and iii). In certain embodiments, the method comprises evaluating: i) and iv). In some embodiments, the method comprises evaluating: ii) and iii). In certain embodiments, the method comprises evaluating: ii) and iv).

In some embodiments, the method further comprises evaluating (e.g., providing a value for ZMF Factor or Muscle Function in) one or more muscles selected from the:
left or right shoulder flexor (SHDFLEX);
left or right elbow flexion (ELBFLEX);
left or right first interosseous contraction (FDIO)
left or right ankle dorsiflexion (ANKLDOR);
left or right knee extensor (KNEEEXT);
left or right wrist extensor (WRSTEXT);
left or right elbow extensor (ELBEXT);
left or right knee flexor (KNEEFLEX); and
left or right hip flexor (HIPFLEX).

In some embodiments, the method further comprises evaluating (e.g., providing a value for ZMF Factor or Muscle Function in) one or more muscles selected from the:
left or right shoulder flexor (SHDFLEX);
left or right knee extensor (KNEEEXT);
left or right wrist extensor (WRSTEXT);
left or right elbow extensor (ELBEXT);
left or right knee flexor (KNEEFLEX); and
left or right hip flexor (HIPFLEX).

In some embodiments, the method further comprises evaluating (e.g., providing a value for ZMF Factor or Muscle Function in) one or more muscles selected from the:
left or right shoulder flexor (SHDFLEX); and
left or right knee extensor (KNEEEXT).

In some embodiments, the method further comprises evaluating (e.g., providing a value for ZMF Factor or Muscle Function in) one or more muscles selected from the:
left or right elbow extensor (ELBEXT); and
left or right knee extensor (KNEEEXT). In certain embodiments, two, three, four, five, six, seven, eight, nine, or ten, or more, muscles are evaluated.

In some embodiments, the method comprises evaluating (e.g., providing a value for Muscle Function or ZMF Factor in) the following muscles:
left or right first dorsal interosseous (FDIO);
left or right ankle dorsiflexion (ANKLDOR);
left or right shoulder flexor (SHDFLEX); and
left or right knee extensor (KNEEEXT).

In some embodiments, the method comprises evaluating (e.g., providing a value for Muscle Function or ZMF Factor in) the following muscles:
left or right first dorsal interosseous (FDIO);
left or right ankle dorsiflexion (ANKLDOR);
left or right elbow extensor (ELBEXT); and
left or right knee extensor (KNEEEXT). In certain embodiments, a plurality of muscles, e.g., two, three, four, five, six, seven, eight, nine, or ten or more muscles, are evaluated and each muscle of the plurality is one of a contralateral pair.

In some embodiments, the muscle is one which reaches, or is expected to reach, Zero Function at or after a preselected time. In certain embodiments, the muscle is one which reaches, or is expected to reach, Zero Function within 1, 2, 4 or 6 months from a preselected point, e.g., after diagnosis of the disease, after initiation of a treatment, or after completion of a treatment. In some embodiments, the muscle is one which reaches, or is expected to reach, Zero Function after a different preselected muscle reaches or is expected to reach Zero Function. In certain embodiments, the muscle is one which reaches, or is expected to reach, Zero Function after a preselected Sentinel Muscle reaches or is expected to reach Zero Function. In some embodiments, the muscle is one which reaches, or is expected to reach, Zero Function after one or more or all of, the L-FDIO, the R-FDIO, the L-ANKLDOR, and the R-ANKLDOR, reaches or is expected to reach Zero Function.

In certain embodiments, the muscle is one which reaches, or is expected to reach, Zero Function prior to a preselected time. In some embodiments, the muscle is one which reaches, or is expected to reach, Zero Function before a preselected Sentinel Muscle reaches or is expected to reach Zero Function. In some embodiments, the muscle is one which reaches, or is expected to reach, Zero Function before one or more or all of, the L-FDIO, the R-FDIO, the L-ANKLDOR, and the R-ANKLDOR, reaches or is expected to reach Zero Function.

In some embodiments, one of L-FDIO, the R-FDIO, the L-ANKLDOR, and the R-ANKLDOR; and a muscle other than the L-FDIO, the R-FDIO, the L-ANKLDOR, and the R-ANKLDOR, are evaluated. In certain embodiments, a Sentinel Muscle and a muscle other than a Sentinel Muscle are evaluated. In some embodiments, one of the L-FDIO and the R-FDIO, one of the L-ANKLDOR and the R-ANKLDOR, one of the L-ELBEXT and the R-ELBEXT, and one of the L-KNEEEXT and the R-KNEEEXT are evaluated.

In some embodiments, the Muscle Function or ZMF Factor comprises a value for muscle function for the L-FDIO. In some embodiments, T_{ZFV} is based on a value for L-FDIO. In certain embodiments, the ZMF Factor comprises a value for muscle function for the L-FDIO but not R-FDIO. In some embodiments, T_{ZFV} is based on a value for L-FDIO but not R-FDIO. In some embodiments, the ZMF Factor comprises a value for muscle function for the R-FDIO. In some embodiments, T_{ZFV} is based on a value for R-FDIO. In certain embodiments, the ZMF Factor comprises a value for muscle function for the R-FDIO but not L-FDIO. In some embodiments, T_{ZFV} is based on a value for R-FDIO but not L-FDIO. In some embodiments, the ZMF Factor comprises a value for muscle function for the L-ANKLDOR. In some embodiments, the ZMF Factor comprises a value for muscle function for the L-ELBEXT. In some embodiments, the ZMF Factor comprises a value for muscle function for the L-ELBEXT but not R-ELBEXT. In some embodiments, the ZMF Factor comprises a value for muscle function for the R-ELBEXT. In some embodiments, the ZMF Factor comprises a value for muscle function for the R-ELBEXT but not L-ELBEXT. In some embodiments, the ZMF Factor comprises a value for muscle function for the L-KNEEEXT. In some embodiments, the ZMF Factor comprises a value for muscle function for the L-KNEEEXT but not R-KNEEEXT. In some embodiments, the ZMF Factor comprises a value for muscle function for the R-KNEEEXT. In some embodiments, the ZMF Factor comprises a value for muscle function for the R-KNEEEXT but not L-KNEEEXT. In some embodiments, the ZMF Factor comprises a value for muscle function for a FDIO, an ANKLDOR, an ELBEXT, and a KNEEEXT. In some embodiments, the ZMF Factor comprises a value for muscle function for one FDIO, ANKLDOR, ELBEXT, or KNEEEXT, but not the contralateral FDIO, ANKLDOR, ELBEXT, or KNEEEXT. In some embodiments, T_{ZFV} is based on a value for L-ANKLDOR. In certain embodiments, the ZMF Factor comprises a value for muscle function for the L-ANKLDOR but not R-ANKLDOR. In some embodiments, T_{ZFV} is based on a value for L-ANKLDOR but not R-ANKLDOR. In some embodiments, the ZMF Factor comprises a value for muscle function for the R-ANKLDOR. In some embodiments, T_{ZFV} is based on a value for R-ANKLDOR. In certain embodiments, the ZMF Factor comprises a value for muscle function for the R-ANKLDOR but not L-ANKLDOR. In some embodiments, T_{ZFV} is based on a value for R-ANKLDOR but not L-ANKLDOR. In some embodiments, the ZMF Factor comprises a value for muscle function for a FDIO and an ANKLDOR. In some embodiments, T_{ZFV} is based on a value for a FDIO and an ANKLDOR. In certain embodiments, the ZMF Factor comprises a value for muscle function for a FDIO and one ANKLDOR but not the contralateral ANKLDOR. In some embodiments, T_{ZFV} is based on a value for a FDIO and one ANKLDOR but not the contralateral ANKLDOR. In some embodiments, the ZMF Factor comprises a value for muscle function for one FDIO but not the contralateral FDIO and an ANKLDOR. In some embodiments, T_{ZFV} is based on a value for one FDIO but not the contralateral FDIO and an ANKLDOR. In certain embodiments, the ZMF Factor comprises a value for muscle function for one FDIO but not the contralateral FDIO and one ANKLDOR but not the contralateral ANKLDOR. In some embodiments, T_{ZFV} is based on a value for one FDIO but not the contralateral FDIO and one ANKLDOR but not the contralateral ANKLDOR.

In some embodiments, the method comprises providing a value for muscle function for the L-FDIO. In certain embodiments, the method comprises providing a value for muscle function for the L-FDIO but not R-FDIO. In some embodiments, the method comprises providing a value for muscle function for the R-FDIO. In certain embodiments, the method comprises providing a value for muscle function for the R-FDIO but not L-FDIO.

In some embodiments, the method comprises providing a value for muscle function for the L-ANKLDOR. In certain embodiments, the method comprises providing a value for muscle function for the L-ANKLDOR but not R-ANKLDOR. In some embodiments, the method comprises providing a value for muscle function for the R-ANKLDOR. In certain embodiments, the method comprises providing a value for muscle function for the R-ANKLDOR but not L-ANKLDOR. In some embodiments, the method comprises providing a value for muscle function for the R-ELBEXT. In some embodiments, the method comprises providing a value for muscle function for the R-ELBEXT but not L-ELBEXT. In some embodiments, the method comprises providing a value for muscle function for the L-ELBEXT. In some embodiments, the method comprises providing a value for muscle function for the L-ELBEXT but not R-ELBEXT. In some embodiments, the method comprises providing a value for muscle function for the R-KNEEEXT. In some embodiments, the method comprises providing a value for muscle function for the R-KNEEEXT but not L-KNEEEXT. In some embodiments, the method comprises providing a value for muscle function for the L-KNEEEXT. In some embodiments, the method comprises providing a value for muscle function for the L-KNEEEXT but not R-KNEEEXT. In some embodiments, the method comprises providing a value for muscle function for a FDIO, an ANKLDOR, an ELBEXT, and a KNEEEXT. In some embodiments, the method comprises providing a value for muscle function for one FDIO, ANKLDOR, ELBEXT, or KNEEEXT, but not the contralateral FDIO, ANKLDOR, ELBEXT, or KNEEEXT.

In some embodiments, the method comprises providing a value for muscle function for a FDIO and an ANKLDOR.

In certain embodiments, the method comprises providing a value for muscle function for a FDIO and one ANKLDOR but not the contralateral ANKLDOR. In some embodiments, the method comprises providing a value for muscle function for one FDIO but not the contralateral FDIO and an ANKLDOR. In further embodiments, the method comprises providing a value for muscle function for one FDIO but not the contralateral FDIO and one ANKLDOR but not the contralateral ANKLDOR.

In some embodiments, subject is not participating in a clinical trial, e.g., a clinical trial of a treatment for a motor neuron disease, e.g., ALS, e.g., bulbar or non-bulbar ALS.

In certain embodiments, Muscle Function is measured with a device that measures one or more of:

the ability to exert force, e.g., force exerted by a limb or extremity, e.g., a distal limb or extremity, e.g., as determined my manual muscle testing, e.g., as evaluated by the Medical Research Council (MRC) Scale for Muscle Strength in a clinic (e.g., this can be evaluated upon one effort, or upon repeated efforts, e.g., upon repeated efforts within a preselected time period);

peak force, e.g., peak force exerted by a limb or extremity, e.g., a distal limb or extremity (e.g., this can be evaluated upon one effort, or upon repeated efforts, e.g., upon repeated efforts within a preselected time period);

the ability to maintain a force, e.g., ability to maintain force exerted by a limb or extremity, e.g., ability to repeat a force level or repeat a motion by a limb or extremity, e.g., for a preselected time (e.g., this can be evaluated upon one effort, or upon repeated efforts, e.g., upon repeated efforts within a preselected time period);

the ability to assume an anti-gravity position, e.g., the ability to assume an anti-gravity position by a limb or extremity, e.g., a distal limb or extremity, e.g., for a preselected time period (e.g., this can be evaluated upon one effort, or upon repeated efforts, e.g., upon repeated efforts within a preselected time period);

the ability to produce an electrophysiological signal (e.g., this can be evaluated upon once, or upon repeatedly, e.g., repeated measurements within a preselected time period);

transmission of an electrophysiological signal from a neuron/axon to a muscle or muscle cell (e.g., this can be evaluated upon once, or upon repeatedly, e.g., repeated measurements within a preselected time period);

contraction of a muscle (e.g., this can be evaluated upon once, or upon repeatedly, e.g., repeated measurements within a preselected time period);

the muscle's range of motion, e.g., the range of motion demonstrated by a limb or extremity, e.g., a distal limb or extremity (e.g., this can be evaluated upon one effort, or upon repeated efforts, e.g., upon repeated efforts within a preselected time period);

speed attainable by the muscle, e.g., speed attainable of a limb or extremity, e.g., a distal limb or extremity, e.g., with regard to a preselected motion (e.g., this can be evaluated upon one effort, or upon repeated efforts, e.g., upon repeated efforts within a preselected time period); or

acceleration attainable by the muscle (e.g., this can be evaluated upon one effort, or upon repeated efforts, e.g., upon repeated efforts within a preselected time period).

In some embodiments, Muscle Function is measured mechanically. In certain embodiments, muscle function is measured by a dynamometer, e.g., a hand-held dynamometer, or a device comprising a force measurement system such as a strain gauge, piezoelectric sensor, capacitance sensor, accelerometer or other force measurement transducers. In some embodiments, Muscle Function is determined by measurement of electrophysiological signals, e.g., is measured by electromyography, e.g., is measured by a device that detects electrophysiological signals. In some embodiments, Muscle Function is measured by vibromyography. In some embodiments, the force measurement system includes a processor and software to acquire, analyze, record and display force measurements and assist a user with interpretation of the measurements, especially in light of muscle function.

Various aspects and functions described herein may be implemented as specialized hardware or software components executing in one or more computer systems. There are many examples of computer systems that are currently in use. These examples include, among others, network appliances, personal computers, workstations, mainframes, networked clients, servers, media servers, application servers, database servers, and web servers.

Other examples of computer systems may include mobile computing devices (e.g., smart phones, tablet computers, and personal digital assistants) and network equipment (e.g., load balancers, routers, and switches). Examples of particular models of mobile computing devices include iPhones, iPads, and iPod touches running iOS operating system available from Apple, Android devices like Samsung Galaxy Series, LG Nexus, and Motorola Droid X, Blackberry devices available from Blackberry Limited, and Windows Phone devices. Further, aspects may be located on a single computer system or may be distributed among a plurality of computer systems connected to one or more communications networks.

For example, various aspects, functions, and processes may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Consequently, embodiments are not limited to executing on any particular system or group of systems. Further, aspects, functions, and processes may be implemented in software, hardware or firmware, or any combination thereof. Thus, aspects, functions, and processes may be implemented within methods, acts, systems, system elements and components using a variety of hardware and software configurations, and examples are not limited to any particular distributed architecture, network, or communication protocol.

Based on the foregoing disclosure, it should be apparent to one of ordinary skill in the art that the invention is not limited to a particular computer system platform, processor, operating system, network, or communication protocol. Also, it should be apparent that the present invention is not limited to a specific architecture or programming language.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a series of graphs showing the loss of muscle strength over time in patients for each muscle.

FIG. 2 is a series of graphs showing the proportion of patients with zero muscle strength over time for each muscle.

FIGS. 3A and 3B are a series of graphs showing the proportion of patients with zero muscle strength over time for each muscle, for patients with bulbar onset ALS (FIG. 3A) or non-bulbar onset ALS (FIG. 3B).

FIG. 4 is a graph comparing the Kaplan-Meier survival curves generated by measuring zero muscle strength for three muscles to the curve generated by measuring death.

FIG. 5 is a graph comparing the Kaplan-Meier survival curves generated by measuring zero muscle strength for three or four muscles to the curve generated by measuring death.

FIG. 6 is a graph comparing the sensitivity in tracking disease progression between the novel muscle strength endpoint compared to survival and ALSFRS-R endpoints.

FIG. 7 is a graph comparing the sensitivity in tracking disease progression between the muscle strength megascore and ALSFRS-R endpoints.

FIG. 8 is a graph showing the Kaplan-Meier survival curves generated by measuring zero muscle strength in two patient groups: patients with bulbar onset ALS or non-bulbar onset ALS (other).

FIG. 9 is a table of Spearman's correlation coefficients for the decline in strength of individual right side muscles as compared to individual right side muscles. White (lighter coloring) indicates stronger correlation and green (darker coloring) indicates weaker correlation.

FIG. 10 is a pair of graphs showing changes in traditional ALS outcomes (ALSFRS-R at left and survival at right) over a 12-month period for subjects experiencing a zero strength endpoint (zero) and subjects not experiencing a zero strength endpoint (non-zero).

FIG. 11 is a pair of graphs of mean change in strength in individual muscles (right side muscles at top and left side muscles at bottom) over a 12-month period for subjects in the EMPOWER trial.

FIG. 12 is a table listing projected sample size requirements for a 12-month two-treatment study based several endpoints.

FIG. 13 is a table listing Spearman's correlation coefficients for the correlation of baseline strength and rate of decline of strength between corresponding left and right side muscles.

FIG. 14 is a graph plotting the proportion of patients reaching the zero strength endpoint or death over a 12-month period.

FIG. 15 is an image of a subject sitting in Accurate Test of Limb Isometric Strength (ATLIS) equipment.

### DETAILED DESCRIPTION

An advantage of methods described herein is the utilization of the objective time point at which a muscle has lost all or substantially all muscle function to track disease progression and evaluate patients having a disease associated with loss of muscle function with improved sensitivity and increased accuracy. Assessment of time taken to reach zero function, in contrast to other alternative evaluation methods currently available in the art, not only captures critical moment in disease progression but also is not masked or confounded by variables that can affect evaluation of muscle function such as age, gender, physical fitness prior to disease onset, and treatment with agents that can manipulate, e.g., artificially increase, muscle function. For example, assessment of muscle strength by use of z-scores or megascores relies upon normalization in order to combine the strength of different muscles which vastly differ in strength capacity and the kinetics of strength decline over time in the disease setting. Such normalization based on z-scores and megascores obscures the variable strength loss of each individual muscle, therefore resulting in loss of information that can contribute to a more accurate representation of the disease progression. In yet another example, some treatments for diseases described herein can artificially increase muscle function, which can affect or skew the muscle function information acquired from a patient and obscure the accuracy of representing disease progression.

The present disclosure features, at least in part, a novel method for evaluating subjects having a disease or disorder associated with loss of muscle function (e.g., a motor neuron disease, a neuromuscular disease, or a myopathy) by measuring the time of reaching zero strength based on a preselected combination of muscles, and determining when the muscle has zero function (e.g., has lost all or substantially all function). In methods currently used in the art, a measurement of zero muscle function, e.g., zero muscle strength, has not been utilized in tracking disease progression or evaluating patients. Rather, measurements of zero function, e.g., zero muscle strength, have previously been ignored or sometimes treated as missing data due to unmeasurability of muscle strength. As shown herein, utilization of when a muscle has reached zero function in at least one muscle group of a plurality, e.g., a preselected combination, of muscles in methods describe herein results in a highly sensitive method for tracking disease progression and evaluating patients.

The methods described herein provide one or more of the following advantages over traditional endpoints and methods of evaluation (e.g., the ALSFRS-R): i) a muscle-function based test for diseases that are characterized by a loss of function; ii) a very sensitive method to track disease progression; iii) a very sensitive method to track early stage disease; iv) a method that is not confounded by variables such as age, gender, body weight, or other therapeutic regimens that were previously or are concurrently administered; v) ease of measurement; vi) shorter time period required for detecting changes in disease progression or therapeutic efficacy; vii) smaller number of participants required for clinically meaningful results and viii) less susceptible to symptomatic effects.

### Definitions

Unless defined otherwise, all technical and scientific terms used herein have the same meaning as commonly understood by one of ordinary skill in the art to which the invention pertains.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Muscle Function” as that term is used herein refers to the ability of a muscle to respond to stimulus, e.g., nerve stimulus. The ability to respond can be measured in any of a number ways. By way of example, parameters which can serve to evaluate muscle function include the ability to exert force; peak force; the ability to maintain force; the ability to assume an anti-gravity position; range of motion; the ability to attain a certain speed; the ability of the muscle to accelerate, e.g., while in motion; an electrophysiological parameter, e.g., neuron conductivity, neuromuscular junction transmission (e.g., the transmission of an electrical or neurological signal from a neuron directly or indirectly to a muscle cell), or ability of an axon to elicit a contraction of a muscle.

“Sentinel Muscle” as that term is used herein refers to a muscle that is typically useful in the measurements made in methods described herein. These are muscles in which zero function is attained in greater than 90% of patients that have a late stage disease associated loss of muscle function. In one embodiment, a Sentinel Muscle is a muscle in which zero function is attained in greater than 90% of patients that have ALS. Exemplary Sentinel Muscles include the left first dorsal interosseous (L-FDIO); the right first dorsal interosseous (R-FDIO); the left ankle dorsiflexion (L-ANKLDOR); and the right ankle dorsiflexion (R-ANKLDOR).

Other muscles can be suitable as Sentinel Muscles. Suitability can be determined by measuring the T_{ZFV} in a patient population. Any muscle which gives substantially the same result, or has predictability substantially the same as, or better than, L-FDIO, R-FDIO, L-ANKLDOR, or R-ANKLDOR can be used. Examples of muscles which may be suitable as Sentinel Muscles include the abductor pollicus brevis (APB), the adductor digiti minimi (ADM), the flexor digitorum, and the flexor pollicus longus in the hands and the extensor digitorum brevis (EDB), the gastrocnemius, the soleus, and the extensor digitorum longus in the legs. Sentinel Muscles generally comprise distal muscles.

“Upper body” as that term is used herein refers to the region of the body that is above the waist. The waist is the part of the abdomen between the rib cage and hips. In some embodiments or in people with slim bodies, the waist is the narrowest part of the torso. For example, the upper body includes the chest, upper back, shoulders, arms, wrists, fingers, and neck. As used herein, muscles from the upper body includes, e.g., SHDFLEX, ELBFLEX, ELBEXT, WRSTEXT, and FDIO.

“Lower body” as that term is used herein refers to the region of the body that is below the waist. For example, the lower body includes the hips, legs, knees, ankles, and toes. As used herein, muscles from the lower body includes, e.g., HIPFLEX, KNEEFLEX, KNEEEXT, and ANKLDOR.

“Time to Zero Function Value” or “T_{ZFV}”, as the term is used herein, is the time elapsed between a preselected time point or event, e.g., time from onset of disease, time from diagnosis, or time from onset of muscle weakness, e.g., Sentinel Muscle weakness, e.g., anterior tibialis weakness, and the time at which Zero Function Value is reached.

“Zero Muscle Function Factor or ZMF Factor” as that term is used herein, refers to a parameter which represents Muscle Function. ZMF Factor comprises a measure or evaluation of Muscle Function for at least one muscle, but may include additional components, e.g., a constant, or a value for Muscle Function of another muscle, but the additional components do not obscure the ability of the ZMF Factor to identify Zero Function in at least one selected muscle, e.g., in a FDIO or an ANKLDOR or both a FDIO and a ANKLDOR.

“Zero Function Value”, as that term is used herein, refers to a value for Muscle Function or ZMF Factor which is within a preselected range of zero, and in an embodiment is zero. The preselected range is a range, which for diagnostic, prognostic, or clinical purposes is essentially equal or similar in effect, predictive, diagnostic, prognostic, or evaluative function, to zero. The range can be determined, e.g., empirically, for specific muscles, specific embodiments of Muscle Function, and specific devices or methods of measuring Muscle Function.

In embodiments, the preselected range of zero is between 0 and a near-zero value, e.g., the value of 95%, 99%, 99.5%, reduction of the baseline level of muscle function in a subject. In some embodiments, the near-zero value is at least 90%. In some embodiments, the near zero value is at least 95%. In some embodiments, the near zero value is at least 99%. In some embodiments, the near zero value is between 90% and 95%. In some embodiments, the near zero value is between 95% and 99%. A near zero value is a value which has substantially the same predictive value as zero. By way of example, if the preselected range of zero is between 0 and a near-zero value that is a 95% reduction of the baseline muscle function level of 100 N (kg*m/s), then the preselected range of zero is between 0 and 5 N. In such embodiments, the baseline level of muscle function is established at the first assessment of muscle function, e.g., at the onset of disease, at diagnosis of the disease, or at the initiation of a clinical study.

“Zero Function”, as used herein, refers to the status of a muscle that has Zero Function Value for Muscle Function, or ZMF. In embodiments, zero function indicates when the muscle has lost all, or substantially all, muscle function. In embodiments where the muscle strength is the parameter that is measured, zero function can also be referred to as zero muscle strength.

As used herein, the terms “treat”, “treatment” and “treating” refer to the reduction or amelioration of the progression, severity and/or duration of a disorder (e.g., ALS), or the amelioration of one or more symptoms (preferably, one or more discernible symptoms) of a disorder resulting from the administration of one or more therapies. In specific embodiments, the terms “treat”, “treatment” and “treating” refer to the amelioration of at least one measurable physical parameter of a disorder, such as muscle function. In other embodiments the terms “treat”, “treatment” and “treating” refer to the inhibition of the progression of a disorder, either physically by, e.g., stabilization of a discernible symptom, physiologically by, e.g., stabilization of a physical parameter, or both.

As used herein, the term “patient” or “subject” typically refers to a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; that will be or has been the object of treatment, observation, and/or experiment. When the term is used in conjunction with administration of a compound or drug, then the patient has been the object of treatment, observation, and/or administration of the compound or drug.

“Symptom”, as used herein, refers to any signs or symptoms of any disease associated with loss of muscle function as described herein. Exemplary symptoms include muscle weakening, muscle atrophy, or loss of function in one or more muscles. Symptoms of ALS include, for example, muscle weakness and atrophy can occur on both sides of the body, spasticity, spasms, muscle cramps, fasciculations, slurred or nasal speech, loss of the ability to breathe due to failure of the muscles of the diaphragm and chest wall to function properly, and/or cognitive problems involving word fluency, decision-making, and memory.

### Measuring Muscle Function

Provided herein are methods for measuring muscle function, which can be particularly useful for tracking disease progression, evaluating patients, and as an endpoint in clinical studies.

Muscle function refers to the ability of a muscle to respond to a stimulus. For example, the ability of a muscle to respond to nerve stimulus, e.g., the transmission of an electrical or neurological signal (e.g., neurotransmitter) from a nerve cell directly or indirectly to a muscle cell. The ability of a muscle respond to a stimulus can be measured in any of a number ways. By way of example, parameters which can serve to evaluate muscle function include any of the following or a combination thereof:

the ability to exert force, e.g., force exerted by a limb or extremity, e.g., a distal limb or extremity (e.g., this can be evaluated upon one effort, or upon repeated efforts, e.g., upon repeated efforts within a preselected time period);

peak force, e.g., peak force exerted by a limb or extremity, e.g., a distal limb or extremity (e.g., this can be evaluated upon one effort, or upon repeated efforts, e.g., upon repeated efforts within a preselected time period);

the ability to maintain force, e.g., ability to maintain force exerted by a limb or extremity, e.g., ability to repeat a force level or repeat a motion by a limb or extremity, e.g., for a preselected time (e.g., this can be evaluated upon one effort, or upon repeated efforts, e.g., upon repeated efforts within a preselected time period);

the ability to assume an anti-gravity position, e.g., the ability to assume an anti-gravity position by a limb or extremity, e.g., a distal limb or extremity, e.g., for a preselected time period (e.g., this can be evaluated upon one effort, or upon repeated efforts, e.g., upon repeated efforts within a preselected time period);

range of motion, e.g., the range of motion demonstrated by a limb or extremity, e.g., a distal limb or extremity (e.g., this can be evaluated upon one effort, or upon repeated efforts, e.g., upon repeated efforts within a preselected time period);

the ability to attain a certain speed, e.g., speed attainable of a limb or extremity, e.g., a distal limb or extremity, e.g., with regard to a preselected motion (e.g., this can be evaluated upon one effort, or upon repeated efforts, e.g., upon repeated efforts within a preselected time period);

the ability of the muscle to accelerate, e.g., while in motion (e.g., this can be evaluated upon one effort, or upon repeated efforts, e.g., upon repeated efforts within a preselected time period); or

an electrophysiological parameter, e.g., neuron conductivity, neuromuscular junction transmission (e.g., the transmission of an electrical or neurological signal from a neuron directly or indirectly to a muscle cell), or ability of an axon to elicit a contraction of a muscle (e.g., this can be evaluated upon once, or upon repeatedly, e.g., repeated measurements within a preselected time period).

The methods described herein include measuring muscle function and determining or assessing when the muscle has lost all or substantially all function. As disclosed herein, a muscle that has lost all or substantially all function is determined to have zero function. Muscle function can be measured in any of the assays or devices known in the art or as described herein.

In embodiments where no muscle function is observed or measured by the assays or devices described herein, the muscle is considered to have zero function. For example, in embodiments where the muscle is measured by dynamometry, when no muscle force is detected by dynamometry, the muscle is assigned a Zero Function Value. In embodiments where the patient cannot move or position the muscle to be tested into the position for testing, the muscle is considered to have zero function, e.g., is assigned a Zero Function Value.

In some embodiments, a muscle that has lost substantially all function may still have some very low, but detectable amount of function. In the methods described herein, such muscles that have lost substantially all function are considered to have zero function, e.g., assigned a Zero Function Value, and are considered in the evaluation of patients and tracking of disease progression in the same manner as the muscles that have no detectable muscle function as described herein. Muscles that have lost substantially all function, and are considered herein to have zero function, may have muscle function within a preselected range of zero. In embodiments, a preselected range of zero is between 0, e.g., as detected by an assay or device described herein, and a near-zero value. A near-zero value, as used herein, is relative to a baseline level of muscle function that is determined at a given timepoint, e.g., at the diagnosis of a patient, at the initiation of a clinical study, or at onset of the disease or a particular symptom of the disease. In embodiments, the near-zero value is a 95%, 96%, 97%, 98%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98% or 99.99%, or more, reduction of the baseline level of muscle function. By way of example, if the near-zero value that is a 95% reduction of the baseline muscle function level of 100 N (kg*m/s), then the near-zero value is 5 N. In such an embodiment, the preselected range of zero is between 0 and 5 N. In embodiments, the preselected range of zero can differ depending on the disease, the stage of the disease when the baseline level of muscle function is determined, or the muscle tested.

### Techniques for Measuring Muscle Function

Muscle function, or a parameter thereof, can be measured by any of various techniques known in the art or described herein. In some embodiments, the parameters involving muscle strength, e.g., the ability of a muscle to exert force, the peak force of a muscle, the ability to maintain force, the ability to assume an anti-gravity position, the range of motion that can be attained by a muscle, or the ability of a muscle to attain a certain speed or to accelerate, can be measured by mechanical means as described herein, such as devices or apparatuses that are designed to measure the output of strength or force. In other embodiments, the parameters involving an electrophysiological parameter of muscle function, e.g., neuron conductivity, neuromuscular junction transmission (e.g., the transmission of an electrical or neurological signal from a neuron directly or indirectly to a muscle cell), or ability of an axon to elicit a contraction of a muscle, can be measured by methods that measure electrochemical or neurochemical signals that are transmitted from a nerve, to a muscle, or in a muscle during contraction.

Dynamometry can be used to measure muscular strength or endurance. Dynamometry is the measurement of force or power. In embodiments, dynamometry can be used to measure the amount of external force produced by a muscle or muscle group during an isometric contraction. In embodiments, muscle strength can be quantified by force or moment of force, produced by a single, isometric, maximum voluntary contraction (MVC). Power is the rate of doing work. For a muscle contraction, power is the ability of a muscle to do work quickly. At the muscle tendon level, it is measured by taking the product of the rate of muscle shortening or lengthening times the muscle force. Functionally, muscle power can be measured by dynamometers capable of measuring force dynamically while simultaneously measuring or controlling the velocity of the movement. Isokinetic dynamometers, such as the Kin Com™ and Cybex™, can control the velocity of the movement and measure, via a strain gauge force transducer, the force applied. The KinCom can measure both concentric and eccentric contractions, as well as, isometric contractions and a special type of isotonic contraction. Force may be measured with any type of appropriate transducers including, but not limited to a dynamometer, e.g., a hand-held dynamometer, or a device comprising a force measurement system such as a strain gauge, piezoelectric sensor, capacitance sensor, accelerometer or other force measurement transducers. In embodiments, hand-held dynamometers (HHDs) can be used to measure muscle strength. The advantages of using a HHD include that it is easy to use, portable, highly reliable, and is widely used clinical and research settings. However, HHD assessments depend to some extent on the strength of the examiner; such limitations can be overcome by using a fixed system (e.g., ATLIS). One example of an HHD is the microFET2 Hand Held Dynamometer. Other dynamometers can also be used in the methods described herein, e.g., a fixed dynamometer or Drachman's dynamometer.

In embodiments, Accurate Test of Limb Isometric Strength (ATLIS) may be used to measure muscle strength (See, e.g., Andres, P L et al. Validation of a New Strength Measurement Device For ALS Clinical Trials. Muscle Nerve 2012, hereby incorporated by reference in its entirety). ATLIS is based on the Tufts Quantitative Neuromuscular Evaluation (TQNE) method. ATLIS employs specialized equipment in which a patient sits while testing muscles at specific positions. The ATLIS method and equipment ensures that patient positioning and examiner technique are not sources of variability (inherent to techniques such as HHD). In a study on the reliability and validity of data from 20 healthy adults and 10 ALS patients, the mean absolute variation between tests was 8.6%, intraclass correlation coefficients for each muscle group were high (0.82-0.99), the Pearson correlation coefficient of mean ATLIS and TQNE scores was 0.90, and a subject survey demonstrated high user acceptance of ATLIS. Electromyography (EMG) can be used to evaluate the electrical signals elicited by a muscle. An electromyograph is used to perform electropmyography, and the resulting waveform that records the electrical activity of a muscle is referred to as an electromyogram. An electromyograph detects the electrical potential generated by muscle cells when these cells are electrically or neurologically activated. In one embodiment, an electromyograph comprises electrodes, e.g., at least two, that can be placed on the surface of a muscle, and a processing system that detects the electrical signal of the muscle via the placed electrodes. In an embodiment, the electrodes are surface electrodes that measure the electrical activity of the muscle directly below the site at which the surface electrodes are placed. In embodiments, surface EMG is limited due to lack of deep muscles reliability. In embodiments, for measuring deep muscles, intramuscular wires can be used to detect an EMG signal.

In embodiments, EMG is used to assess how well a muscle can be activated. Several analytical methods for determining muscle activation are commonly used depending on the application. In an embodiment, the maximum voluntary contraction (MVC) is performed on the muscle that is being tested. In embodiments, MVC is used as a means of analyzing peak force and force generated by the Sentinel Muscles.

EMG signals are essentially made up of superimposed motor unit action potentials (MUAPs) from several motor units. For a thorough analysis, the measured EMG signals can be decomposed into their constituent MUAPs. MUAPs from different motor units tend to have different characteristic shapes, while MUAPs recorded by the same electrode from the same motor unit are typically similar. Notably, MUAP size and shape depend on where the electrode is located with respect to the tested muscle and so can appear to be different if the electrode moves position.

When the muscle is voluntarily contracted, action potentials begin to appear. As the strength of the muscle contraction is increased, more and more muscle fibers produce action potentials. When the muscle is fully contracted, there should appear a disorderly group of action potentials of varying rates and amplitudes (a complete recruitment and interference pattern). The electrical source is the muscle membrane potential of about −90 mV. Measured EMG potentials range between less than 50 μV and up to 20 to 30 mV, depending on the muscle under observation. Typical repetition rate of muscle motor unit firing is about 7-20 Hz, depending on the size of the muscle, previous axonal damage and other factors.

In some embodiments, motor unit number estimation (MUNE), a standard neurophysiologic technique, can be used to measure muscle function. MUNE is a technique to estimate the number of motor units active in a muscle. Decreases in the number of active motor units are expected in patients having a disease associated with loss of muscle function, e.g., a motor neuron disease or related neuromuscular disorder, e.g., ALS or SMA. Motor unit number index (MUNIX) is a method for assessment of number and size of motor units using compound muscle action potential (CMAP) and surface electromyographic interference pattern (SIP). MUNIX may also be used to measure muscle strength and/or function (see, e.g., Nandedkar et al. Muscle Nerve. 2010 November; 42(5):798-807. doi: 10.1002/mus.21824, hereby incorporated by reference).

In some embodiments, electrical impedance myography (EIM) may be used to measure muscle strength and/or function. EIM utilizes bioimpedance to assess the status of a muscle. Electrodes are placed on a tissue of interest and apply a current. The current generates a voltage, measured by two further electrodes positioned within the span of the current providing electrodes. The voltage is proportional to the resistance of the tissue. Biological tissue comprises lipid bilayers which act as capacitors (building up and releasing charge) and this means the tissue also displays reactance. Resistance and reactance make the voltage sinusoidal and the current sinusoidal out of phase with one another; this phase shift can be used to compare muscle states, e.g., the phase shift in healthy and the phase shift in disease muscle. Tissue resistance, in general, rises as disease progression decreases muscle mass and increases connective tissue and fat mass, while reactance decreases with reduced muscle mass and atrophy. In embodiments, EIM measuring the frequency dependence of current, voltage, and phase information from muscles can also be informative of muscle function and strength. In embodiments, EIM measuring electrical anisotropy (e.g., the directional dependence of current, voltage, and phase, e.g., across muscle vs. along muscle) can also be informative of muscle function and strength.

In some embodiments, axonal excitability may be used to measure muscle strength and/or function. See, e.g., Vucic et al. Clin Neurophysiol. 2006 July; 117(7):1458-66. Epub 2006 Jun. 8, hereby incorporated by reference.

Mechanomyography (MMG) measures the mechanical signal observable from the surface of the muscle when the muscle is contracted. The output of the mechanomyography is a mechanomyogram. At the onset of muscle contraction, gross changes in the muscle shape can cause a large peak in the mechnomyogram. Subsequent vibrations that appear in the mechanomyogram after the initial peak are due to oscillations of the muscle fibers at the resonance frequency of the muscle. Mechanomyography includes acoustic myography (AMG), phonomygraphy (PMG), sound myography, surface mechanomyography, and vibromyography (VMG). Devices that perform mechanomyography can include microphone elements, contact transducers (piezoelectric devices or sensors), accelerometers, recording systems, and/or a combination of sensors attached to the skin.

Acoustic myography, phonomyography, and sound myography assess, e.g., record, the sounds produced during muscle contraction. In embodiments, the sounds produced during muscle contraction become louder as the contraction force increases. Vibromyography measures the vibration signals generated during muscle contraction. Methods and a vibromyograph device are described, e.g., in EP2988675.

In embodiments, transcutaneous oximetry (PtcO2) can be used to measure muscle function. PtcO2 is the non-invasive measurement or determination of the partial pressure of oxygen and/or carbon dioxide in the capillaries of a patient's tissues (e.g., affected tissues, e.g., affected muscle tissue). PtcO2 devices include the SenTec Digital Monitor, and comprise electrodes placed on a patient's skin (e.g., skin of the face or earlobe). While non-invasive, discrepancy can occur between arterialized blood and transcuatenous values, with the latter usually higher when discrepancy occurs. Arterial blood sampling may be more accurate but more invasive.

In embodiments, serum creatinine levels can be used to measure disease progression in patients having a disease associated with loss of muscle function, e.g., a motor neuron disease or related neuromuscular disorder, e.g., ALS or SMA. Decreases in serum creatinine levels correlate with ALSFRS-R decline and loss of walking ability. In an embodiment, serum creatinine levels can be measured using 24 hour urinary excretion measurement (e.g., creatinine in the collected urine) as a proxy; this measurement reflects muscle mass. Other methods and/or devices that may be useful for measuring muscle function are further described in U.S. Pat. Nos. 7,493,812, 6,792,801, 7,470,233, US20120172763, and DE 2912981. Further methods that may be useful for measuring muscle function include magnetoencephalography (MEG).

### Selection of Muscles

Provided herein are methods for selecting muscles that are useful in the methods for tracking disease progression and evaluating patients as described herein. Also provided herein are muscles and combinations of muscles that are useful in the methods for tracking disease progression and evaluating patients as described herein.

In one aspect, the method described herein involves measuring the function of one or more, e.g., one, two, three, four, five six, seven, eight, nine, ten, or more, muscles. In embodiments, a plurality of muscles is measured, e.g., two, three, four, five, six, seven, eight, nine or ten or more, muscles are measured. In an embodiment, the function of one muscle is measured. In an embodiment, the function of two muscles is measured. In an embodiment, the function of three muscles is measured. In an embodiment, the function of four muscles is measured. In an embodiment, the function of five muscles is measured. In an embodiment, the function of six muscles is measured. In an embodiment, the function of seven muscles is measured. In an embodiment, the function of eight muscles is measured. In an embodiment, the function of nine muscles is measured. In an embodiment, the function of ten muscles is measured.

There are three types of muscle: skeletal or striated muscle, cardiac muscle, and smooth muscle. Muscle action can be classified as being either voluntary or involuntary. Cardiac and smooth muscles contract without conscious thought and are termed involuntary, whereas the skeletal muscles contract upon command. Skeletal muscles in turn can be divided into fast and slow twitch fibers.

Muscles useful to the methods described herein are skeletal muscles and the action, e.g., contraction, of such muscles is voluntary. Skeletal muscles from any part of the body can be useful to the methods described herein. In embodiments, the muscle is located in a limb of the body, and includes, but is not limited to, a muscle of the arm, elbow, wrist, finger, leg, knee, ankle, or toe. In embodiments, the muscle is located in the core of the body, and includes, but is not limited to, a muscle of the trunk, abdomen, back, chest, shoulder, or hip. Examples of muscles whose function can be measured as described herein include, but are not limited to: shoulder flexion (SHDFLEX), elbow flexion (ELBFLEX), hip flexion (HIPFLEX), knee flexion (KNEEFLEX), elbow extension (ELBEXT), knee extension (KNEEEXT), wrist extension (WRSTEXT), first interosseous contraction (FDIO), and ankle dorsiflexion (ANKLDOR or ANKLE). Other muscles that can be measured by methods as described herein can also be used together or in combination with any of the other aforementioned muscles.

In embodiments, the function of a particular muscle on the left side of the body is measured or the function of a particular muscle on the right side of the body is measured. In some embodiments, the function of a muscle only on one side of the body is measured, and the function of the corresponding muscle on the contralateral side of the body is not measured. In other words, the function of a muscle is measured unilaterally (e.g., only on side of the body), and not bilaterally (e.g., not on both sides of the body). Even so, measurement of muscle function is not restricted to unilateral measurements.

By way of example, when a muscle on the left side is measured, measurement of the corresponding muscle on the contralateral, or right, side of the body may not be needed for a determination of disease progression. By way of another example, when a muscle of the right side of the body is measured, measurement of the corresponding muscle on the contralateral, or left, side of the body may not be needed for a determination of disease progression. Analysis of muscle strength data from two large ALS clinical trials as described in Example 1 demonstrated that when a muscle lost function, e.g., reached zero function, on one side of the body, the function of the corresponding muscle on the opposite, e.g., contralateral, side of the body rapidly lost function. Without wishing to be bound by any theory, it is believed that once a muscle on one side of the body reaches zero function, the measurement and determination of zero function of the contralateral muscle is not necessary for a determination of disease progression. In an embodiment, measurement of a muscle from one side is sufficient to evaluate a bilateral muscle group, regardless of whether the contralateral muscle is measured.

In embodiments where more than one muscle is measured, e.g., two, three, four, five, six, seven, eight, nine, or ten or more muscles, at least one muscle is from the upper body and at least one muscle is from the lower body. By way of example, when two muscles are measured, one muscle is from the upper body and the other muscle is from the lower body. In another example, when three muscles are measured, one muscle is from the upper body, another muscle is from the lower body, and the third muscle can be from either the upper body or the lower body. Without wishing to be bound by theory, because the disease associated with muscle function loss spreads from a particular onset site or region to the rest of the body, combinations of muscles from both the upper and lower body are believed to provide a more sensitive endpoint or capacity to track disease progression for individual patients. In embodiments in which the function of more than one muscle is measured, at least one muscle from the upper body is measured and at least one muscle from the lower body is measured.

In embodiments where more than one muscle is measured, e.g., two, three, four, five, six, seven, eight, nine, or ten or more muscles, at least one muscle (e.g., one, two, three, four, five, six, seven, eight, nine, or ten or more muscles) is selected with a preference for distal muscles (e.g., distal to the torso) over proximal muscles (e.g., proximal to the torso). By way of example, when three muscles are selected, one, two or three of the muscles is a distal muscle in a disease affected limb, onset site, or region (e.g., FDIO or ANKLDOR). Without wishing to be bound by theory, combinations of muscles including at least one distal muscle of a disease affected limb, onset site, or region are believed to provide a more sensitive endpoint or capacity to track disease progression for individual patients.

A combination of muscles measured in any of the methods described herein can be selected or optimized based on the duration of assessment, the disease associated with loss of muscle function loss, or sensitivity of muscle to disease progression. In some embodiments, the combination of muscle group can be selected or optimized based on the duration of assessment, e.g., the length of time for which a clinical study is performed. In such embodiments, a combination of muscles can be selected or optimized based on 1 month, 2 months, 3 months, 4 months, 5 months, 6 months, 7 months, 8 months, 9 months, 10 months, 12 months, 13 months, 14 months, 15 months, 16 months, 17 months, 18 months, 19 months, 20 months, 21 months, 22 months, 23 months, 24 months, 30 months, 36 months, 42 months, or 48 months. The duration of assessment can influence the choice of muscle or combination of muscles that are measured by the methods described herein. In embodiments in which the duration of assessment is shorter, e.g., shorter than 15 months, shorter than 12 months, shorter than 9 months, or shorter than 6 months, at least one of the muscles measured in any of the methods described herein is a Sentinel Muscle. In embodiments in which the duration of assessment is longer, e.g., longer than 6 months, longer than 9 months, longer than 12 months, longer than 15 months, longer than 18 months, or longer than 24 months, one or more of the muscles measured in any of the methods described herein takes a longer time, e.g., longer than a Sentinel Muscle, to reach zero function. In embodiments, a combination of muscles measured for a longer duration of assessment would comprise more muscles that take longer to reach zero function, e.g., than a Sentinel Muscle, than the combination of muscles used to measure a shorter duration of assessment. In other embodiments, a combination of muscles measured for a shorter duration of assessment would comprise less muscles that take longer to reach zero function, e.g., than a Sentinel Muscle, than the combination of muscles used to measure a longer duration of assessment. In other embodiments, a combination of muscles measured for a longer duration of assessment would comprise more muscles that take longer to reach zero function, than the combination of muscles used to measure a shorter duration of assessment.

In some embodiments, the muscle function of one muscle is measured. In one embodiment, the muscle measured is selected from the group comprising SHDFLEX, ELBFLEX, HIPFLEX, KNEEFLEX, ELBEXT, KNEEEXT, WRSTEXT, FDIO and ANKLDOR.

In one embodiment, the muscle measured is the first dorsal interosseous (FDIO). In one embodiment, the muscle function of FDIO on one side of the body is measured, but the muscle function of the FDIO on the contralateral side of the body is not measured. By way of example, the function of the left FDIO (FDIO-L) is measured or the function of the right FDIO (FDIO-R) is measured. In one embodiment, the function of the left FDIO is measured. In one embodiment, the function of the right FDIO is measured.

In one embodiment, the muscle measured is the first dorsal interosseous (FDIO). In one embodiment, the muscle function of FDIO on both sides of the body is measured. By way of example, the function of the left FDIO (FDIO-L) is measured and the function of the right FDIO (FDIO-R) is measured.

In one embodiment, the muscle measured is the ankle dorsiflexion (ANKLDOR). In one embodiment, the muscle function of ANKLDOR on one side of the body is measured, but the muscle function of ANKLDOR on the contralateral side of the body is not measured. By way of example, the function of the left ANKLDOR (ANKLDOR-L) is measured or the right ANKLDOR (ANKLDOR-R) is measured. In one embodiment, the function of the left ANKLDOR is measured. In one embodiment, the function of the right ANKLDOR is measured.

In some embodiments, the muscle function of two muscles is measured. In one embodiment, the muscle function of each of the two muscles is measured on one side of the body, but not the contralateral side of the body. In one embodiment, one of the muscles measured is from the upper body and the other muscle measured is from the lower body.

In one embodiment, the muscle function of the FDIO and the ANKLDOR is measured. In one embodiment, the muscle function of the FDIO and the ANKLDOR on one side of the body is measured, but the muscle function of the FDIO and the ANKLDOR on the contralateral side of the body is not measured. In such embodiments, the FDIO and ANKLDOR muscles that are measured do not need to be on the same side of the body. In one embodiment, the function of the right or the left FDIO and the right or the left ANKLDOR is measured. In one embodiment, the function of the right FDIO and the right ANKLDOR is measured. In one embodiment, the function of the right FDIO and the left ANKLDOR is measured. In one embodiment, the function of the left FDIO and the right ANKLDOR is measured. In one embodiment, the function of the left FDIO and the left ANKLDOR is measured.

In another embodiment, the muscle function of FDIO and another muscle, e.g., a muscle selected from the group consisting of SHDFLEX, ELBFLEX, HIPFLEX, KNEEFLEX, ELBEXT, KNEEEXT, WRSTEXT, and ANKLDOR, is measured. In an embodiment, the muscle function of FDIO and another muscle from the lower body, e.g., a muscle selected from the group consisting of HIPFLEX, KNEEFLEX, KNEEEXT, and ANKLDOR, is measured. In another embodiment, the muscle function of ANKLDOR, and another muscle, e.g., a muscle selected from the group consisting of SHDFLEX, ELBFLEX, HIPFLEX, KNEEFLEX, ELBEXT, KNEEEXT, WRSTEXT, and ANKLDOR, is measured. In an embodiment, the muscle function of ANKLDOR and another muscle from the upper body, e.g., a muscle selected from the group consisting of SHLDFLEX, ELBFLEX, ELBEXT, WRSTEXT, and FDIO, is measured.

In some embodiments, the muscle function of three muscles is measured. In one embodiment, the muscle function of each of the three muscles on one side of the body is measured, but the corresponding muscles on the contralateral side of the body are not measured.

In one embodiment, the muscle function of the FDIO and two other muscles, e.g., selected from the group consisting of SHDFLEX, ELBFLEX, HIPFLEX, KNEEFLEX, ELBEXT, KNEEEXT, WRSTEXT, and ANKLDOR, is measured. In such embodiments, one of the two other muscles is a muscle from the lower body. In one embodiment, the muscle function of the ANKLDOR and two other muscles, e.g., selected from the group consisting of SHDFLEX, ELBFLEX, HIPFLEX, KNEEFLEX, ELBEXT, KNEEEXT, WRSTEXT, and FDIO, is measured. In one embodiment, the muscle function of the FDIO, ANKLDOR, and another muscle, e.g., selected from the group consisting of SHDFLEX, ELBFLEX, HIPFLEX, KNEEFLEX, ELBEXT, KNEEEXT, and WRSTEXT, is measured. In such embodiments, one of the two other muscles is a muscle from the upper body.

In one embodiment, the three muscles measured can be any of the following combinations: FDIO, ANKLDOR, and ELBEXT; FDIO, ANKLDOR, and ELBFLEX; FDIO, ANKLDOR, and SHLDFLEX; FDIO, ANKLDOR, and WRSTEXT; FDIO, ANKLDOR, and KNEEEXT; or FDIO, ANKLDOR, and KNEEFLEX.

In some embodiments, the muscle function of four muscles is measured. In one embodiment, the muscle function of each of the four muscles on one side of the body is measured, but muscle function of the corresponding muscles on the contralateral side of the body is not measured.

In one embodiment, the muscle function of the FDIO and three other muscles, e.g., selected from the group consisting of SHDFLEX, ELBFLEX, HIPFLEX, KNEEFLEX, ELBEXT, KNEEEXT, WRSTEXT, and ANKLDOR, is measured. In one embodiment, the muscle function of the ANKLDOR and three other muscles, e.g., selected from the group consisting of SHDFLEX, ELBFLEX, HIPFLEX, KNEEFLEX, ELBEXT, KNEEEXT, WRSTEXT, and FDIO, is measured. In one embodiment, the muscle function of the FDIO, ANKLDOR, and two other muscles, e.g., selected from the group consisting of SHDFLEX, ELBFLEX, HIPFLEX, KNEEFLEX, ELBEXT, KNEEEXT, and WRSTEXT, is measured.

In one embodiment, the four muscles measured can be any of the following combinations: FDIO, ANKLDOR, SHDFLEX, and KNEEEXT; FDIO, ANKLDOR, SHDFLEX, and KNEEFLEX; FDIO, ANKLDOR, SHDFLEX, and HIPFLEX; FDIO, ANKLDOR, SHDFLEX, and WRSTEXT; FDIO, ANKLDOR, KNEEEXT, and KNEEFLEX; FDIO, ANKLDOR, KNEEEXT, and WRSTEXT; FDIO, ANKLDOR, KNEEEXT, and HIPFLEX; FDIO, ANKLDOR, KNEEFLEX, and WRSTEXT; FDIO, ANKLDOR, KNEEEXT, and WRSTEXT; FDIO, ANKLDOR, KNEEEXT, and HIPFLEX; FDIO, ANKLDOR, WRSTEXT, and HIPFLEX; FDIO, ANKLDOR, ELBEXT, and KNEEEXT.

In some embodiments, the muscle function of five muscles is measured. In one embodiment, the muscle function of each of the five muscles on one side of the body is measured, but the muscle function of each of the five muscles on the contralateral side of the body is not measured. In one embodiment, the muscle function of the FDIO and four other muscles, e.g., selected from the group consisting of SHDFLEX, ELBFLEX, HIPFLEX, KNEEFLEX, ELBEXT, KNEEEXT, WRSTEXT, and ANKLDOR, is measured. In one embodiment, the muscle function of the ANKLDOR and four other muscles, e.g., selected from the group consisting of SHDFLEX, ELBFLEX, HIPFLEX, KNEEFLEX, ELBEXT, KNEEEXT, WRSTEXT, and FDIO, is measured. In one embodiment, the muscle function of the FDIO, ANKLDOR, and three other muscles, e.g., selected from the group consisting of SHDFLEX, ELBFLEX, HIPFLEX, KNEEFLEX, ELBEXT, KNEEEXT, and WRSTEXT is measured.

In some embodiments, the muscle function of six muscles is measured. In one embodiment, the muscle function of each of the six muscles on the contralateral side of the body is not measured. In one embodiment, the muscle function of the FDIO and five other muscles, e.g., selected from the group consisting of SHDFLEX, ELBFLEX, HIPFLEX, KNEEFLEX, ELBEXT, KNEEEXT, WRSTEXT, and ANKLDOR, is measured. In one embodiment, the muscle function of the ANKLDOR and five other muscles, e.g., selected from the group consisting of SHDFLEX, ELBFLEX, HIPFLEX, KNEEFLEX, ELBEXT, KNEEEXT, WRSTEXT, and FDIO, is measured. In one embodiment, the muscle function of the FDIO, ANKLDOR, and four other muscles, e.g., selected from the group consisting of SHDFLEX, ELBFLEX, HIPFLEX, KNEEFLEX, ELBEXT, KNEEEXT, and WRSTEXT is measured.

In some embodiments, the muscle function of seven muscles is measured. In one embodiment, the muscle function of each of the seven muscles is measured on one side of the body, but the muscle function of each of the seven muscles on the contralateral side of the body is not measured. In one embodiment, the muscle function of the FDIO and six other muscles, e.g., selected from the group consisting of SHDFLEX, ELBFLEX, HIPFLEX, KNEEFLEX, ELBEXT, KNEEEXT, WRSTEXT, and ANKLDOR, is measured. In one embodiment, the muscle function of the ANKLDOR and six other muscles, e.g., selected from the group consisting of SHDFLEX, ELBFLEX, HIPFLEX, KNEEFLEX, ELBEXT, KNEEEXT, WRSTEXT, and FDIO, is measured. In one embodiment, the muscle function of the FDIO, ANKLDOR, and five other muscles, e.g., selected from the group consisting of SHDFLEX, ELBFLEX, HIPFLEX, KNEEFLEX, ELBEXT, KNEEEXT, and WRSTEXT, is measured.

In some embodiments, the muscle function of eight muscles is measured. In one embodiment, the muscle function of each of the eight muscles is measured on one side of the body, but the muscle function of each of the eight muscles on the contralateral side of the body is not measured. In one embodiment, the muscle function of the FDIO and seven other muscles, e.g., selected from the group consisting of SHDFLEX, ELBFLEX, HIPFLEX, KNEEFLEX, ELBEXT, KNEEEXT, WRSTEXT, and ANKLDOR, is measured. In one embodiment, the muscle function of the ANKLDOR and seven other muscles, e.g., selected from the group consisting of SHDFLEX, ELBFLEX, HIPFLEX, KNEEFLEX, ELBEXT, KNEEEXT, WRSTEXT, and FDIO, is measured. In one embodiment, the muscle function of the FDIO, ANKLDOR, and six other muscles, e.g., selected from the group consisting of SHDFLEX, ELBFLEX, HIPFLEX, KNEEFLEX, ELBEXT, KNEEEXT, and WRSTEXT, is measured.

In some embodiments, the muscle function of nine muscles is measured. In one embodiment, the muscle function of each of the nine muscles is measured on one side of the body, but the muscle function of each of the nine muscles on the contralateral side of the body is not measured. In one embodiment, the muscle function of the FDIO and eight other muscles, e.g., selected from the group consisting of SHDFLEX, ELBFLEX, HIPFLEX, KNEEFLEX, ELBEXT, KNEEEXT, WRSTEXT, and ANKLDOR, is measured. In one embodiment, the muscle function of the ANKLDOR and eight other muscles, e.g., selected from the group consisting of SHDFLEX, ELBFLEX, HIPFLEX, KNEEFLEX, ELBEXT, KNEEEXT, WRSTEXT, and FDIO, is measured. In one embodiment, the muscle function of the FDIO, ANKLDOR, and seven other muscles, e.g., selected from the group consisting of SHDFLEX, ELBFLEX, HIPFLEX, KNEEFLEX, ELBEXT, KNEEEXT, and WRSTEXT, is measured.

In some embodiments more than ten muscles are used in the analysis.

### Uses

Provided herein are methods for assessing motor function, evaluating patients, and tracking disease progression for diseases associated with loss of muscle function. Diseases associated with loss of muscle function, e.g., ALS, can be challenging to track in an accurate or sensitive manner. In many diseases associated with loss of muscle function, e.g., ALS, use of muscle strength measurements has not provided a more sensitive or accurate means to evaluate patients or track disease progression than current or traditional endpoints, such as survival or subject analyses such as survey or questionnaire-based endpoints, e.g., ALSFRS-R. Here, for the first time, a more sensitive and accurate method is described that utilizes muscle strength measurements, specifically, the measurement of when a muscle has reached zero function.

The present disclosure features a method for evaluating a patient having a disease associated with loss of muscle function as described herein. Evaluation of a patient includes, but is not limited to, one or more of any of the following: tracking the progression of the disease; identifying a pattern, e.g., an abnormal or a normal pattern, of disease progression; evaluating a patient for zero function of a muscle, e.g., assigning a Zero Function Value or a ZMF value to a muscle; determining if a muscle in a patient has reached zero function; upon the determination of the muscle having zero function, classifying the patient as having reached zero function for a muscle; upon the determination of the muscle having not reached zero function, classifying the patient as having not reached zero function for a muscle; determining the prognosis of the disease; predicting the outcome of the disease; predicting the survival of a patient; evaluating the effectiveness of a treatment; and/or determining a preferred treatment regimen.

In embodiments, tracking the progression of a disease associated with loss of muscle function comprises measuring the muscle function of a preselected plurality of muscles multiple times. In embodiments, the measuring of muscle function is performed over the course of the disease or a clinical study, e.g., at every visit to the doctor's office or at set intervals of time, e.g., once a month, once every two months, once every three months, or once every six months.

In embodiments, responsive to the value for muscle function, e.g., determining that zero function has been reached or assigning a Zero Function Value to a muscle of a subject, the method further includes classifying, selecting, modifying prognosis or treatment or making a prediction about the subject. For example, the methods described herein are useful for determining a course of action with respect to treatment regimen options.

In embodiments, the evaluation of the patient is performed at a preselected amount of time. A preselected amount of time includes, but is not limited to: at the initial diagnosis of the disease associated with muscle function loss, e.g., ALS; at initial administration of a treatment for the disease associated with muscle function loss, e.g., ALS; at the initiation into a clinical trial; at any interval specified by a clinical trial, e.g., once a month, once every two months, once every three months, or once every 6 months; at the completion of a clinical trial; at a modification of treatment for the disease associated with muscle function loss, e.g., ALS; at the abandonment or conclusion of a treatment for the disease associated with muscle function loss, e.g., ALS; upon failure to respond to a treatment for the disease associated with muscle function loss, e.g., ALS; at the presentation of a preselected symptom, or disease stage; or prior to presentation of a preselected symptom.

In embodiments, the methods described herein are particularly useful for screening or identifying a candidate therapeutic and determining therapeutic effect. The present methods can also be used for animal models in clinical or research settings for identifying candidate therapeutics for a disease associated with loss of muscle function, as described herein. As in the case of ALS, the present methods provide an opportunity to identify therapeutics that have therapeutic benefit where ones have not previously been identified, due in part to the insensitivity of the traditional endpoints that have been used in clinical studies for ALS to date. As discussed herein, the present methods disclosed are more sensitive and more accurate that current endpoints used in clinical studies, and would reveal therapeutic benefits of candidate therapies that may not be revealed using the current endpoints.

The present disclosure features a method that can be administered and interpreted by clinicians or other skilled persons who are not necessarily skilled or experience in evaluating diseases associated with loss of muscle function. Also, the present disclosure features a method that provides a means for evaluating patients at any point during disease progression, and provides an accurate and objective assessment of the disease that is not obscured by biases or nuances of the patient population or the individual patient.

In an embodiment, the state of a subject's disease, the progression of a subject's disease, the response of a subject to treatment, the response to a change in treatment, or another parameter related to the subject's disease, e.g., expressed as a function of Muscle Function or for a Zero Muscle Function Factor (ZMF Factor), can be correlated with another factor. Examples of such factors include genotype, phenotype, gender, age, family history, disease history, an environmental factor, mobility, or a selected activity. The sensitivity of the evaluations described herein can allow for the discovery of new associations, e.g., a genotype, environmental factor or other factor, not previously recognized to be correlated with the disease. Factors found to be correlated with disease can be used in the classification, evaluation, diagnosis, prognosis, or treatment, of a subject.

In an embodiment, the state of a subject's disease, the progression of a subject's disease, the response of a subject to treatment, the response to a change in treatment, or another parameter related to the subject's disease is correlated with genotype. In an embodiment, genotype refers to the diploid combination of alleles for a given genetic polymorphism. A homozygous subject carries two copies of the same allele and a heterozygous subject carries two different alleles.

Certain genotypes are currently known to be associated with ALS. For example, certain mutations in the SOD1, TDP43, and FUS genes are known to cause ALS, while other genetic flaws such as expansions in the C9ORF72 or ataxin 2 gene, extra copies of the SMN1 gene and repeat expansion mutations in the NIPA1 gene are associated with a higher risk of developing the disease. The methods described herein can advantageously provide for the identification of new genes correlated with presence or severity of ALS.

In some embodiments, the state of a subject's disease, the progression of a subject's disease, the response of a subject to treatment, the response to a change in treatment, or another parameter related to the subject's disease is correlated with phenotype. In an embodiment phenotype refers to any observable or otherwise measurable physiological, morphological, biological, biochemical or clinical characteristic of an organism.

As described herein, phenotypic symptoms of ALS include weakness and atrophy of the bulbar muscles (muscles that control speech, swallowing, and chewing), including loss of strength and the ability to move their arms and legs, and to hold the body upright. Muscle weakness and atrophy can occur on both sides of the body. Other symptoms include spasticity, spasms, muscle cramps, fasciculations, slurred or nasal speech, loss of the ability to breathe due to failure of the muscles of the diaphragm and chest wall to function properly, and/or cognitive problems involving word fluency, decision-making, and memory. The methods described herein can advantageously provide for the identification of new phenotypes correlated with presence or severity of ALS.

In some embodiments, the state of a subject's disease, the progression of a subject's disease, the response of a subject to treatment, the response to a change in treatment, or another parameter related to the subject's disease is correlated with family history. At present, about 10% of cases are considered “familial ALS” (FALS). In these cases, more than one person in the family has ALS. The methods described herein can advantageously provide for the identification of new correlations between family history and ALS.

In some embodiments, the state of a subject's disease, the progression of a subject's disease, the response of a subject to treatment, the response to a change in treatment, or another parameter related to the subject's disease is correlated with an environmental factor. Environmental factors can be any aspect of the environment that influences the individual, directly or indirectly. Exemplary environmental factors include, but are not limited to, air quality, water quality, diet, soil quality, chemical exposure, radiation exposure, and geographic area. The methods described herein can advantageously provide for the identification of new environmental factors correlated with presence or severity of ALS.

In some embodiments, the state of a subject's disease, the progression of a subject's disease, the response of a subject to treatment, the response to a change in treatment, or another parameter related to the subject's disease is correlated with a selected activity. Exemplary selected activities include occupation, hobbies, use of drugs/alcohol/cigarettes, consumption of a particular food or beverage, and exercise. For example, the risk of ALS has been found to be lower among people who drink alcohol than among those who do not. Smokers have a significantly higher risk of developing ALS than people who have never smoked. The methods described herein can advantageously provide for the identification of new correlations between selected activities and presence or severity of ALS.

In some embodiments, the state of a subject's disease, the progression of a subject's disease, the response of a subject to treatment, the response to a change in treatment, or another parameter related to the subject's disease, e.g., expressed as a function of Muscle Function or for a Zero Muscle Function Factor (ZMF Factor), can be correlated with one factor, e.g., genotype, phenotype, family history, disease history, an environmental factor, mobility, or a selected activity. In some embodiments, the state of a subject's disease, the progression of a subject's disease, the response of a subject to treatment, the response to a change in treatment, or another parameter related to the subject's disease, e.g., expressed as a function of Muscle Function or for a Zero Muscle Function Factor (ZMF Factor), can be correlated with two factors, e.g., genotype, phenotype, family history, disease history, an environmental factor, mobility, or a selected activity. In some embodiments, the state of a subject's disease, the progression of a subject's disease, the response of a subject to treatment, the response to a change in treatment, or another parameter related to the subject's disease, e.g., expressed as a function of Muscle Function or for a Zero Muscle Function Factor (ZMF Factor), can be correlated with three or more factors, e.g., genotype, phenotype, family history, disease history, an environmental factor, mobility, or a selected activity.

Diseases Associated with Loss of Muscle Function

Provided herein are methods that are useful in diagnosing, prognosing, and tracking the progression of a disease or disorder associated with loss of muscle function as described herein. The methods described herein may be particularly useful for diseases or disorders in which a symptom is muscle weakening, muscle atrophy, or loss of function in one or more muscles. In embodiments, the disease or disorder associated with muscle function loss involves the loss of the ability of a neuron, e.g., a motor neuron, to elicit a response in a muscle, e.g., a muscle cell, which can also be referred to herein as loss of neuromuscular junction transmission. For example, diseases and disorders associated with muscle function loss can include, but are not limited to, motor neuron diseases (MND), spinal muscular atrophy (SMA), Kennedy's disease, post-polio syndrome (PPS), myopathies, neuromuscular disease, or related disorders. In an embodiment, the disease or disorder associated with muscle function loss can be a neurological or neurodegenerative disorder, in which the patient also experiences loss of muscle function, muscle weakness, or muscle atrophy.

In embodiments, the disease or disorder associated with muscle function loss is a motor neuron disease (MND) or related motor neuron disorder. Examples of MNDs and related disorders include, but are not limited to, amyotrophic lateral sclerosis (ALS), primary lateral sclerosis (PLS), progressive muscular atrophy (PMA), progressive bulbar palsy (PBP), and pseudobulbar palsy. MNDs and related disorders generally are neurological disorders that selectively affect the motor neurons, thereby resulting in reduced or loss of control of voluntary muscles of the body.

Amyotrophic lateral sclerosis (ALS), also called Lou Gehrig's disease or classical motor neuron disease, is a progressive, ultimately fatal disorder that disrupts signals to all voluntary muscles. Both upper and lower motor neurons are affected. In some embodiments, ALS is bulbar ALS. In some embodiments, ALS is non-bulbar ALS. In bulbar ALS, symptoms are typically first noticed in the face and mouth. In non-bulbar ALS, symptoms are typically first noticed in the limbs. Approximately 80% of ALS cases are non-bulbar ALS and approximately 20% of ALS cases are bulbar ALS cases. Symptoms of ALS include weakness and atrophy of the bulbar muscles (muscles that control speech, swallowing, and chewing), including loss of strength and the ability to move their arms and legs, and to hold the body upright. Muscle weakness and atrophy can occur on both sides of the body. Other symptoms include spasticity, spasms, muscle cramps, fasciculations, slurred or nasal speech, loss of the ability to breathe due to failure of the muscles of the diaphragm and chest wall to function properly, and/or cognitive problems involving word fluency, decision-making, and memory. Treatments for ALS can include pharmacological and non-pharmacological treatments, and combinations thereof. Non-pharmacological treatments include managing symptoms with physical, occupational, speech, respiratory and nutritional therapy; the application of heat to relieve muscle cramping; exercise to maintain muscle strength and function; and nerve stimulation. Pharmacological treatments include medications which increase survival time and/or aid quality of life by maintaining muscle function, e.g., riluzole; botulinum toxin to treat jaw spasms or drooling; amitriptyline, glycopyolate, atropine or botulinum injections into the salivary glands to treat excess saliva; antispasticity agents baclofen (Lioresal®), benzodiazepines, and tizanidine (Zanaflex®); dextromethorphan and quinidine to reduce pseudobulbar affect; antidepressants to treat depression; and/or anticonvulsants and nonsteroidal anti-inflammatory drugs to treat pain. In embodiments, the treatment includes one or more non-pharmacological treatments. In embodiments, the treatment includes one or more pharmacological treatments. In embodiments, the treatment includes one or more pharmacological treatments in combination with one or more non-pharmacological treatments. Pharmacological and/or non-pharmacological treatments administered in combination can be administered concurrently, overlapping, and/or sequentially.

Primary lateral sclerosis (PLS) affects the upper motor neurons of the arms, legs, and face. It occurs when specific nerve cells in the motor regions of the cerebral cortex (the thin layer of cells covering the brain which is responsible for most high-level brain functions) gradually degenerate, causing the movements to be slow and effortful. The disorder often affects the legs first, followed by the body trunk, arms and hands, and, finally, the bulbar muscles. Symptoms include slow and/or slurred speech; stiff, clumsy, slow and weak legs and arms that can lead to an inability to walk or carry out tasks requiring fine hand coordination; difficulty with balance that can lead to falls; and/or pseudobulbar affect and/or an overactive startle response. The symptoms progress gradually over years, leading to progressive stiffness and clumsiness of the affected muscles. PLS is sometimes considered a variant of ALS, but the major difference is the sparing of lower motor neurons, the slow rate of disease progression, and normal lifespan.

Progressive muscular atrophy (PMA) is marked by slow but progressive degeneration of only the lower motor neurons. It largely affects men, with onset earlier than in other MNDs. Symptoms include weakness typically seen first in the hands that then spreads into the lower body, where it can be severe. Other symptoms may include muscle wasting, clumsy hand movements, fasciculations, and muscle cramps. The trunk muscles and respiration may become affected. Exposure to cold can worsen symptoms. PMA can develop into ALS.

Progressive bulbar palsy (PBP), also called progressive bulbar atrophy, involves the brain stem—the bulb-shaped region containing lower motor neurons needed for swallowing, speaking, chewing, and other functions. Symptoms include pharyngeal muscle weakness (involved with swallowing), weak jaw and facial muscles, progressive loss of speech, tongue muscle atrophy, limb weakness with both lower and upper motor neuron signs, increased risk of choking and aspiration pneumonia, and/or outbursts of laughing or crying, e.g., emotional lability.

Pseudobulbar palsy, which shares many symptoms of progressive bulbar palsy, is characterized by degeneration of upper motor neurons that transmit signals to the lower motor neurons in the brain stem. Symptoms include progressive loss of the ability to speak, chew, and swallow; progressive weakness in facial muscles leading to an expressionless face; development of a gravelly voice and an increased gag reflex; and outbursts of laughing or crying. The tongue may become immobile and unable to protrude from the mouth.

Treatments for motor neuron diseases include managing symptoms with physical, occupational, speech, respiratory and nutritional therapy; botulinum toxin to treat jaw spasms or drooling; amitriptyline, glycopyolate, atropine or botulinum injections into the salivary glands to treat excess saliva; antispasticity agents baclofen (Lioresal), benzodiazepines, and tizanidine (Zanaflex); dextromethorphan and quinidine to reduce pseudobulbar affect; antidepressants to treat depression; and/or anticonvulsants and nonsteroidal anti-inflammatory drugs to treat pain.

Spinal muscular atrophy (SMA) is a hereditary disease affecting the lower motor neurons. It is an autosomal recessive disorder caused by defects in the gene SMN1, which makes a protein that is important for the survival of motor neurons (SMN protein). In SMA, insufficient levels of the SMN protein lead to degeneration of the lower motor neurons, producing weakness and wasting of the skeletal muscles. This weakness is often more severe in the trunk and upper leg and arm muscles than in muscles of the hands and feet. SMA in children is classified into three types, based on ages of onset, severity, and progression of symptoms. All three types are caused by defects in the SMN1 gene.

SMA type I, also called Werdnig-Hoffmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing. Some children also develop scoliosis (curvature of the spine) or other skeletal abnormalities. Affected children never sit or stand and the vast majority usually die of respiratory failure before the age of 2.

Symptoms of SMA type II, the intermediate form, usually begin between 6 and 18 months of age. Children may be able to sit but are unable to stand or walk unaided, and may have respiratory difficulties.

Symptoms of SMA type III (Kugelberg-Welander disease) appear between 2 and 17 years of age and include abnormal gait; difficulty running, climbing steps, or rising from a chair; and a fine tremor of the fingers. The lower extremities are most often affected. Complications include scoliosis and joint contractures—chronic shortening of muscles or tendons around joints, caused by abnormal muscle tone and weakness, which prevents the joints from moving freely. Individuals with SMA type III may be prone to respiratory infections, but with care may have a normal lifespan.

Congenital SMA with arthrogryposis (persistent contracture of joints with fixed abnormal posture of the limb) is a rare disorder. Symptoms include severe contractures, scoliosis, chest deformity, respiratory problems, unusually small jaws, and drooping of the upper eyelids.

Treatments for SMA include physical therapy, occupational therapy, and rehabilitation to improve posture, prevent joint immobility, and slow muscle weakness and atrophy; stretching and strengthening exercises to reduce spasticity, increase range of motion, and keep circulation flowing; therapy for speech, chewing, and swallowing difficulties; applying heat to relieve muscle pain; assistive devices such as supports or braces, orthotics, speech synthesizers, and wheelchairs; muscle relaxants such as baclofen, tizanidine, and the benzodiazepines to reduce spasticity; botulinum toxin to treat jaw spasms or drooling; amitriptyline, glycopyolate, atropine or botulinum injections into the salivary glands to treat excess saliva; and/or antidepressants to treat depression.

Kennedy's disease, also known as progressive spinobulbar muscular atrophy, is an X-linked recessive disease caused by mutations in the gene for the androgen receptor. Daughters of individuals with Kennedy's disease are carriers and have a 50 percent chance of having a son affected with the disease. The onset of symptoms is variable and the disease may first be recognized between 15 and 60 years of age. Symptoms include weakness and atrophy of the facial, jaw, and tongue muscles, leading to problems with chewing, swallowing, and changes in speech; muscle pain and fatigue in arm and leg muscles closest to the trunk of the body that develops over time, with muscle atrophy and fasciculations; and sensory loss in the feet and hands. Affected individuals may have enlargement of the male breasts or develop noninsulin-dependent diabetes mellitus. The course of the disorder varies but is generally slowly progressive. Treatment includes physical therapy and rehabilitation to slow muscle weakness and atrophy.

Post-polio syndrome (PPS), including Post-Polio Muscular Atrophy (PPMA), is a condition that can strike polio survivors decades after their recovery from poliomyelitis. Polio is an acute viral disease that destroys motor neurons. Many people who are affected early in life recover and develop new symptoms many decades later. After acute polio, the surviving motor neurons expand the amount of muscle that each controls. PPS and Post-Polio Muscular Atrophy (PPMA) are thought to occur when the surviving motor neurons are lost in the aging process or through injury or illness. Symptoms include fatigue, slowly progressive muscle weakness, muscle atrophy, fasciculations, cold intolerance, muscle and joint pain, and difficulty breathing, swallowing, and/or sleeping. Symptoms are more frequent among older people and those individuals most severely affected by the earlier disease. Some individuals experience only minor symptoms, while others develop muscle atrophy that may be mistaken for ALS. PPS is not usually life threatening. Treatment for PPS includes nonfatiguing exercises to improve muscle strength and reduce tiredness, prednisone, intravenous immunoglobulin, and/or lamotrigine.

In embodiments, the disease or disorder associated with muscle function loss is a myopathy. Myopathy is a common term for a muscle disease that is unrelated to any disorder of innervation or neuromuscular junction, with a wide range of possible etiologies. The primary symptom is muscle weakness due to dysfunction of muscle fiber. Other symptoms of myopathy can include muscle cramps, stiffness, and spasm.

The myopathies can be divided into hereditary and acquired disorders. Hereditary group encompasses muscular dystrophies, congenital myopathies, metabolic myopathies, mitochondrial myopathies, as well as myotonias and channelopathies. Acquired myopathies include inflammatory, endocrine and toxic myopathies.

Muscular dystrophies comprise a heterogeneous group of hereditary illnesses affecting both children and adults, with at least 30 different genes responsible for the disease development. Symptoms include muscle wasting and weakness, with elevated levels of creatine kinase (CK). The diseases show a dystrophic pattern (i.e. degenerative pattern with necrosis and extensive fibrosis) and an involvement of the central nervous system. Treatment includes physical therapy, respiratory therapy, speech therapy, orthopedic appliances used for support, corrective orthopedic surgery, corticosteroids to slow muscle degeneration, anticonvulsants to control seizures and some muscle activity, immunosuppressants to delay some damage to dying muscle cells, and antibiotics to fight respiratory infections.

Congenital myopathies comprise a genetically and clinically heterogeneous group of conditions, originally classified according to unique morphological changes observed in the muscle tissue. No necrotic or degenerative changes are present in congenital myopathies (in contrast to muscular dystrophy) and CK levels are often normal. This group of myopathies includes nemaline myopathy, central core disease, X-linked myotubular myopathy and centronuclear myopathy. Treatments include orthopedic treatments, physical therapy, occupational therapy, and/or speech therapy

Metabolic myopathies comprise a diverse group of disorders which arise as a result of defects in cellular energy metabolism, including the breakdown of fatty acids and carbohydrates to generate adenosine triphosphate. Three main categories of metabolic myopathies are fatty acid oxidation defects, glycogen storage diseases, and mitochondrial disorders due to respiratory chain impairment.

Mitochondrial myopathies are also a large group of variegated disorders resulting from primary dysfunction of the mitochondrial respiratory chain and subsequently causing muscle disease. This group of illnesses has a myriad of different phenotypes and genetic etiologies, and can frequently present with multi-system dysfunction. Symptoms of mitochondrial myopathies include muscle weakness or exercise intolerance, heart failure or rhythm disturbances, dementia, movement disorders, stroke-like episodes, deafness, blindness, droopy eyelids, limited mobility of the eyes, vomiting, and seizures. Examples of mitochondrial myopathies include severe Pearson syndrome, Kearns-Sayre syndrome and progressive external ophthalmoplegia which can manifest in late adulthood. Treatments include physical therapy to extend the range of movement of muscles and improve dexterity and vitamin therapies such as riboflavin, coenzyme Q, and carnitine.

Genetic defects in the genes that code for calcium, sodium, potassium and chloride channels in skeletal muscles can result in the periodic paralyses, the nondystrophic myotonias, and the ryanodinopathies. This group of diseases includes myotonia congenita, paramyotonia congenita, hyper and hypokalemic periodic paralysis, potassium-aggravated myotonia, as well as Andersen-Tawil syndrome.

The idiopathic inflammatory myopathies constitute a subset of autoimmune connective tissue diseases primarily affecting the muscle, along with a myriad of extra-muscular manifestations. These conditions can be sub-classified into dermatomyositis, polymyositis and inclusion body myositis, according to differences in clinical and histopathological features. The muscle pathology shows characteristic inflammatory exudates of variable distribution within the muscle fascicle, and there is a variable degree of CK elevation and irritative myopathy.

Muscle weakness and myopathy can also be found in endocrinologic conditions. For example, it is commonly found in acromegaly as a result from a combination of the direct effect of growth hormone excess on muscle, but also from other metabolic derangements as well (such as hypoadrenalism, hypothyroidism or diabetes mellitus). Cushing's disease, characterized by overproduction of hormones by the pituitary and adrenal glands, can also cause myopathy.

Myopathy is also included among the potential side-effects and toxicities associated with the certain lipid lowering agents (such as 3-hydroxy-3-methylglutaryl coenzyme A reductase inhibitors), but also corticosteroids or alcohol.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Identification of a Novel Endpoint Based on Muscle Strength Measures

Muscle strength testing was used as a secondary endpoint in two recent ALS clinical trials, the ceftriaxone study (Cudkowicz et al., “Safety and efficacy of ceftriaxone for amyotrophic lateral sclerosis: a multi-stage, randomized, double-blind, placebo-controlled trial”, Lancet Neurol 2014, 13:1083-91; hereby incorporated by reference in its entirety) and the EMPOWER study (Cudkowicz et al., “Dexpramixpexole versus placebo for patients with amyotrophic lateral sclerosis (EMPOWER): a randomized, double-blind, phase 3 trial”, Lancet Neurol 2013, 12:1059-67; hereby incorporated by reference in its entirety). As described in this example, the results from the muscle strength testing obtained in both of these studies were analyzed in order to identify a new endpoint based on muscle strength measures.

The ceftriaxone study was a multi-stage, randomized, double-blind, placebo-controlled study. Participants of the study had amyotrophic lateral sclerosis, a vital capacity of more than 60% of that predicted for age and height, and symptom duration of less than 3 years. 513 total patients were randomly allocated (2:1) to receive ceftriaxone (2 g or 4 g per day) or placebo for 12 months. The coprimary efficacy endpoints were survival and functional decline, as measured as the slope of ALSFRS-R scores. This study was registered with ClinicalTrials.gov, number NCT00349622. The parameters and findings of this study are further described in Cudkowicz et al., Lancet Neurol 2014, 13:1083-91.

The EMPOWER study was a double-blind, placebo-controlled Phase III clinical trial on dexpramipexole in patients with ALS. Participants of the study were between 18 and 80 years old (with first amyotrophic lateral sclerosis symptom onset 24 months or less before baseline) from 81 academic medical centres in 11 countries. 942 eligible patients were randomly (1:1) allocated to receive twice-daily 150 mg dexpramipexole or matched placebo for 12-18 months. The primary endpoint was the combined assessment of function and survival (CAFS) score, based on changes in the ALSFRS-R total scores and time to death up to 12 months. The primary endpoint was assessed in all participants who received at least one dose and had at least one post-dose ALSFRS-R measurement or died. This study was registered with ClinicalTrials.gov, number NCT01281189. The parameters and findings of this study are further described in Cudkowicz et al., Lancet Neurol, 2013; 12:1059-67.

Muscle strength of each participant was assessed in both the ceftriaxone study and EMPOWER study as a secondary endpoint. Nine muscle groups were tested bilaterally, e.g., left and right of each of: shoulder flexion (SHDFLEX), elbow flexion (ELBFLEX), hip flexion (HIPFLEX), knee flexion (KNEEFLEX), elbow extension (ELBEXT), knee extension (KNEEEXT), wrist extension (WRSTEXT), first interosseous contraction (FDIO), and ankle dorsiflexion (ANKLDOR), for a total of 18 muscles tested. Muscle strength of each participant was measured at multiple timepoints: every 3 months in the ceftriaxone study and every 2 months in the EMPOWER study, beginning at the start of the study for 12 months. For each patient's visit during the trial, strength testing was performed with a patient sitting in a hard backed chair with arm rests. If a patient was too weak to transfer comfortably, they could be tested in their wheelchair. Each muscle was tested with a standard beginning position, usually midway between maximum flexion or extension of the muscle being studied. After the standard position was attained, the evaluator placed the HHD device along the limb, with standard position defined for each muscle. The patient was instructed to steadily increase force against the device until they achieved maximal exertion; during that time, the evaluator attempted to match the patient's force so that the limb did not move. After maximum strength was achieved, the evaluator exerted force against the dynamometer sufficient to overcome the subject's contraction. For very strong muscles (for example, knee extensors) evaluators could occasionally not overcome the patient's strength. This failure was noted on the case report form. For each muscle to be tested, two evaluations were performed separated by at least 15 sec. If the variability of the 2 evaluations was less than 15%, the maximum value was recorded and the evaluator proceeded to the next muscle to be tested. If variability was greater than 15%, a third trial was performed, and the maximum value of the 3 trials was accepted. Order of muscle testing was standardized. Measurements were made in pounds. A value of “0” was assigned to a given muscle if the patient could not assume the testing position due to weakness, or could attain the position but not exert measureable force.

A recent study analyzed the results from the muscle strength assessments from these two clinical studies, normalizing the strength measurements with megascores or z-scores, as described in Shefner et al., “Quantitative Strength Testing in ALS Clinical Trials”, hereby incorporated by reference in its entirety. Comparison between the quantitative strength measurement with ALSFRS-R and vital capacity (VC) showed that ALSFRS-R was more sensitive in tracking disease progression than the quantitative strength measurement. As loss of strength is an intrinsic component of decline in ALS, new ways of analyzing and utilizing strength testing results are needed to provide an assessment or endpoint that is more sensitive than the current standard, ALSFRS-R. The ceftriaxone and EMPOWER study together represent one of the largest cohorts of ALS patients studied over 12 months, and the muscle strength data from these studies allowed for the identification of the use of the “zero” strength measurement to provide a novel endpoint that is more sensitive than ALSFRS-R and survival.

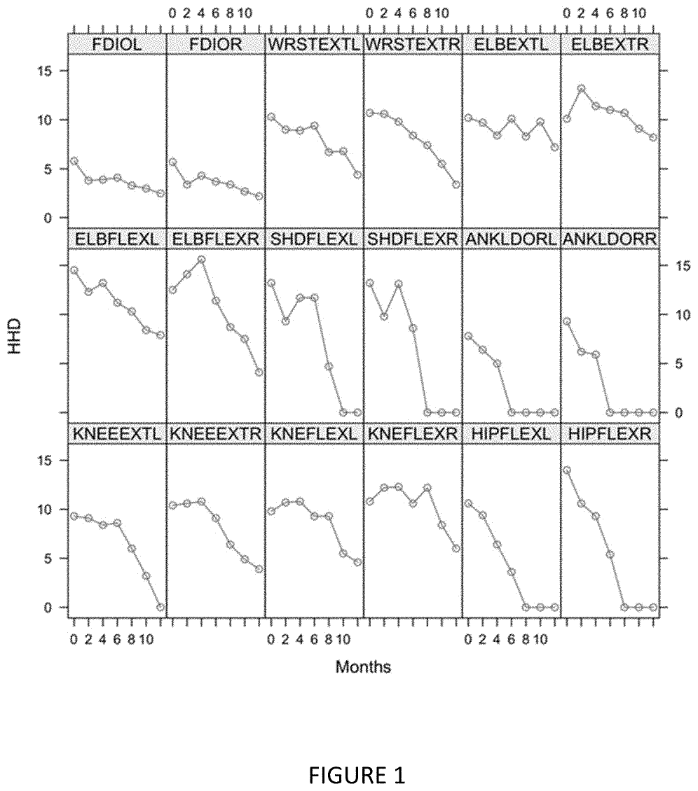

When muscle strength was plotted over time for each individual muscle for any given patient, it was shown that the patient gradually lost muscle strength over time. FIG. 1 shows the muscle strength loss in each tested muscle in one patient. These results show that rates of muscle strength loss are different for different muscles. For example, FDIO and ANKLDOR (left or right) showed the fastest change, losing muscle strength and reaching “zero” the earliest, and KNEEEXT showed the slowest change. These results also show that once a muscle reached “zero” strength, the muscles stayed at “zero” for the rest of the follow-up period.

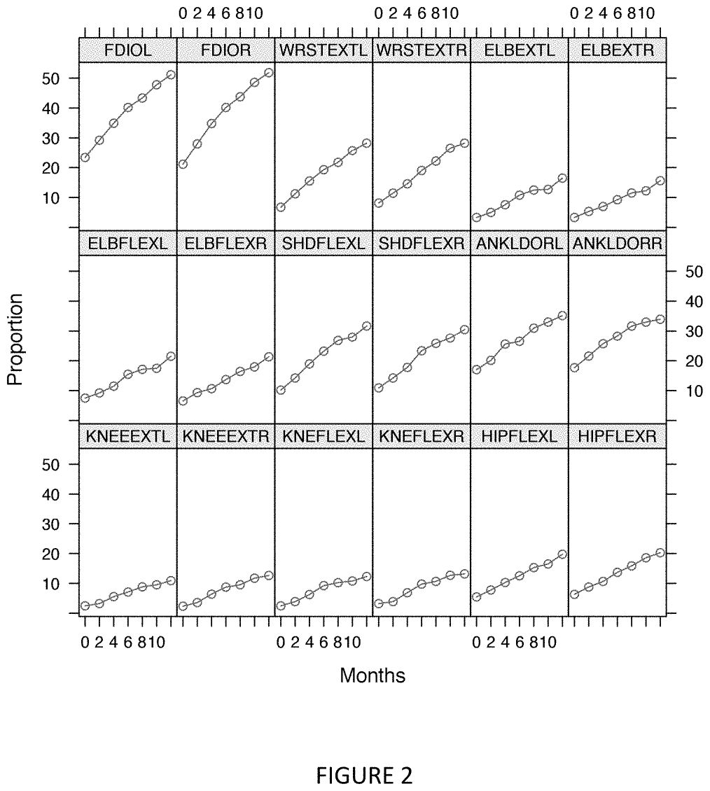

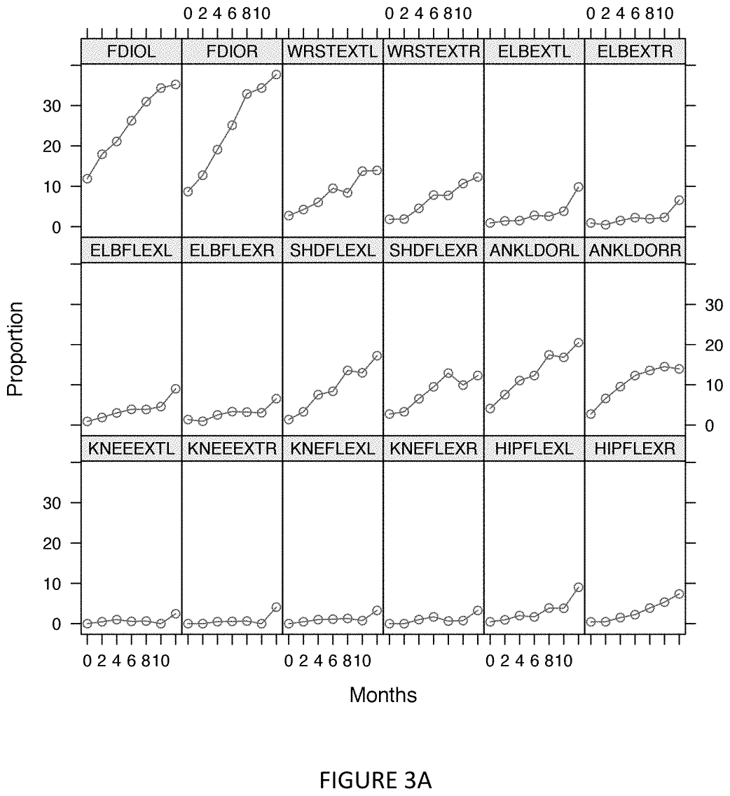

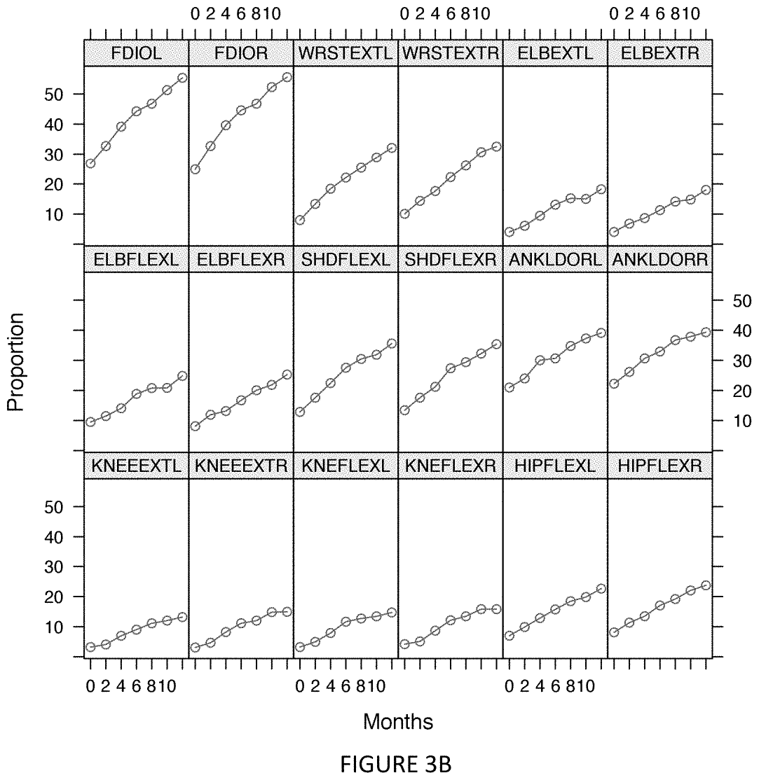

For each tested muscle, the proportion of patients with zero strength measure at each visit was first calculated (FIG. 2). The proportion of patients with zero strength increased over time for each muscle. This analysis showed that the FDIO and ANKLDOR muscle had the largest proportions at each visit among the 18 muscles tested (9 muscles bilaterally). When patients were further stratified into bulbar onset (FIG. 3A) and non-bulbar onset (FIG. 3B), the general trends observed regarding the proportion of patents reaching zero strength for each muscle group was consistent. Further analysis also demonstrated that the overlap was small between patients whose FDIO reached zero strength and those whose ANKLDOR reached zero strength. This suggests random disease onset on upper and lower body regions. Thus, both FDIO and ANKLDOR muscle measurements need to be included in the new endpoint in order to capture patients with different body regions of onset.

Next, for each of the 18 muscles, its earliest time reaching zero strength since study entry was calculated. To be specific, for a particular muscle of a patient, if its strength reached zero during the study follow-up, the earliest time since study entry that the muscle reached zero strength was recorded and the outcome was marked as an “event”. On the other hand, if the muscle never reached zero strength during follow-up, the outcome was marked as “right censored”. In particular, if the muscle already had zero reading at study entry, the outcome was marked as “left censored” and it was not used in the new endpoint.

The bilateral property of reaching zero strength was studied for each muscle. These analyses indicated that for a majority of patients, their left and right side of the same muscle reached zero strength at approximately the same time. This suggests that once one side of a muscle reached zero, the other side would reach zero soon. For this reason, it was determined that once one side of a muscle reached zero strength, the other side of the muscle would typically not be informative at all in tracking disease progression. For example, it may not need to be used unless the initial muscle is unavailable for measurement, e.g., if use of the initial limb is no longer available because of trauma or stroke. Therefore in order for a muscle to be informative, neither side of it had to be nonzero at study entry.

Based on the above analyses, if FDIO and ANKLDOR (e.g., 4 muscles bilaterally) were the only muscles included in the new endpoint, only about 95% of patients had neither side of FDIO or neither side of ANKLDOR reached zero strength at study entry. In other words, there were still 5% of patients who had at least one side of FDIO or ANKLDOR already reached zero at study entry and these patients would not be able to provide an informative outcome during study follow-up. In such cases, the inclusion of more muscles would ensure that each patient could potentially provide an informative muscle strength measure based on the new endpoint.

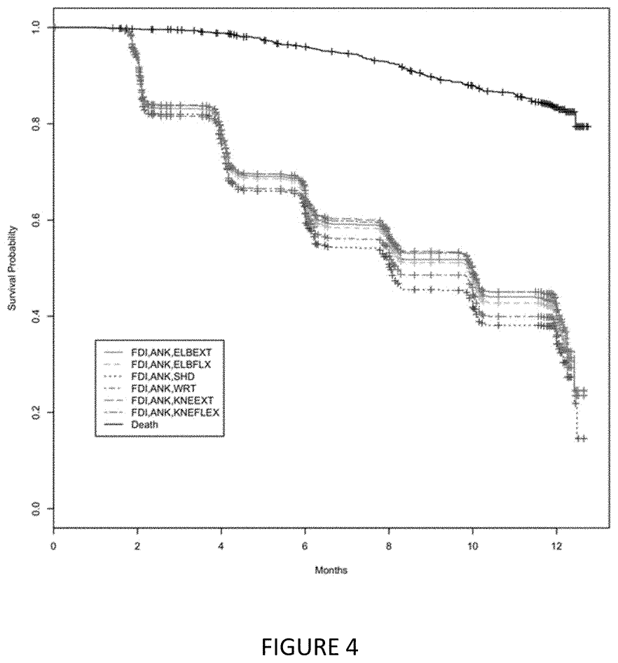

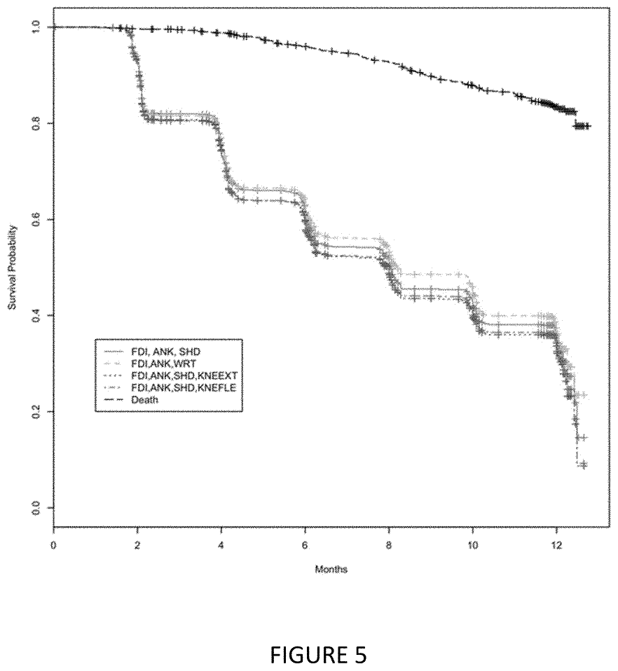

Thus, the Kaplan-Meier curves were generated and plotted for all combinations of 3 muscles, each combination including both FDIO and ANKLDOR, together with the one based on patient survival (FIG. 4). Each muscle combination was ranked based on its separation from the survival curve. The one with the largest separation was the combination of FDIO, ANKLDOR and SHOULDER. This process was repeated by looking at Kaplan-Meier curves based on all combinations of 4 muscles, each including FDIOL, ANKLDOR and SHOULDER (FIG. 5). The one with the largest separation from the survival curve was the combination including FDIO, ANKLDOR, SHOULDER and KNEE EXTENSION. Using this combination, up to 99.7% of the patients in the EMPOWER study can potentially provide an informative outcome. This combination of muscles was further verified using data from the Ceftriaxone study and it showed that up to 99.8% of patients at study entry can potentially provide an outcome. Therefore, new endpoint based on these four muscles is not only sensitive in tracking disease progression but also feasible to be used in ALS clinical trials.

These results shows that a new endpoint can be defined as the earliest time from study entry that one of, for example, the 8 muscles (right/left FDIO, right/left ANKLDOR, right/left SHOULDER, right/left KNEE) reaches zero strength measure when a patient has at least one muscle with neither side having already reached zero at study entry.

### Example 2: Comparison of the Novel Muscle Strength Endpoint to Traditional Endpoints

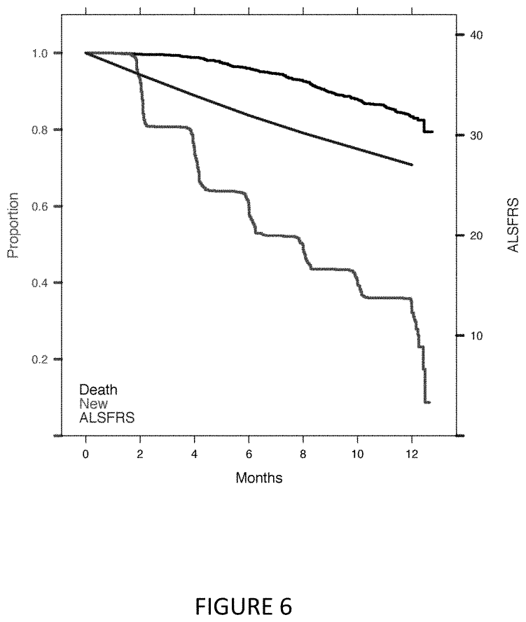

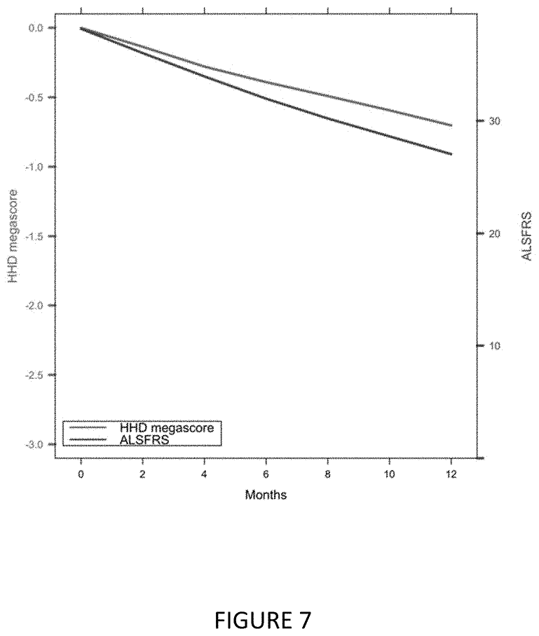

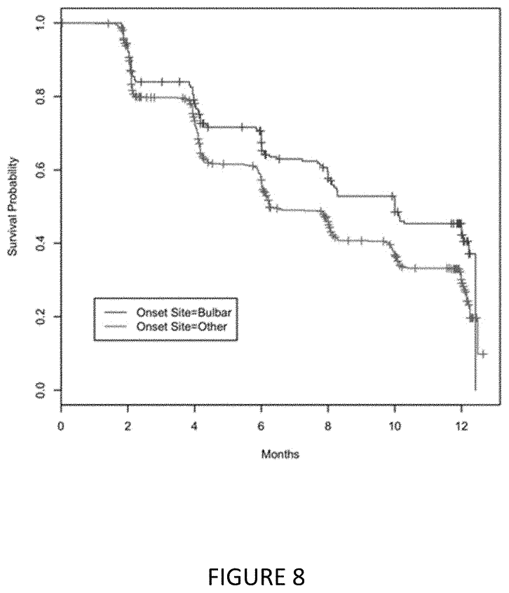

Comparison between the proportion of patients reaching zero strength or death to the ALSFRS-R scale for the same patients is shown in FIG. 6. This comparison shows that the ALSFRS-R is more sensitive than using survival as an endpoint for tracking disease progression. This comparison also shows that the new endpoint utilizing the measurement of zero function (e.g., zero strength) is even more sensitive than ALSFRS-R in tracking disease progression. In contrast, comparison between evaluation by ALSFRS-R or by using megascores generated from muscle strength measurements shows that ALSFRS-R is more sensitive for tracking disease progression (FIG. 7). Thus, although computing megascores involves measuring muscle strength, these results clearly show that the type muscle strength information used, and manipulation of the muscle strength information, is important for determining the timepoint of when a muscle, or a preselected combination of muscles, reaches zero strength is more sensitive than by using megascore that represents a non-zero value of muscle strength.

### Example 3: Evaluating Additional Muscle Strength Endpoint(s)

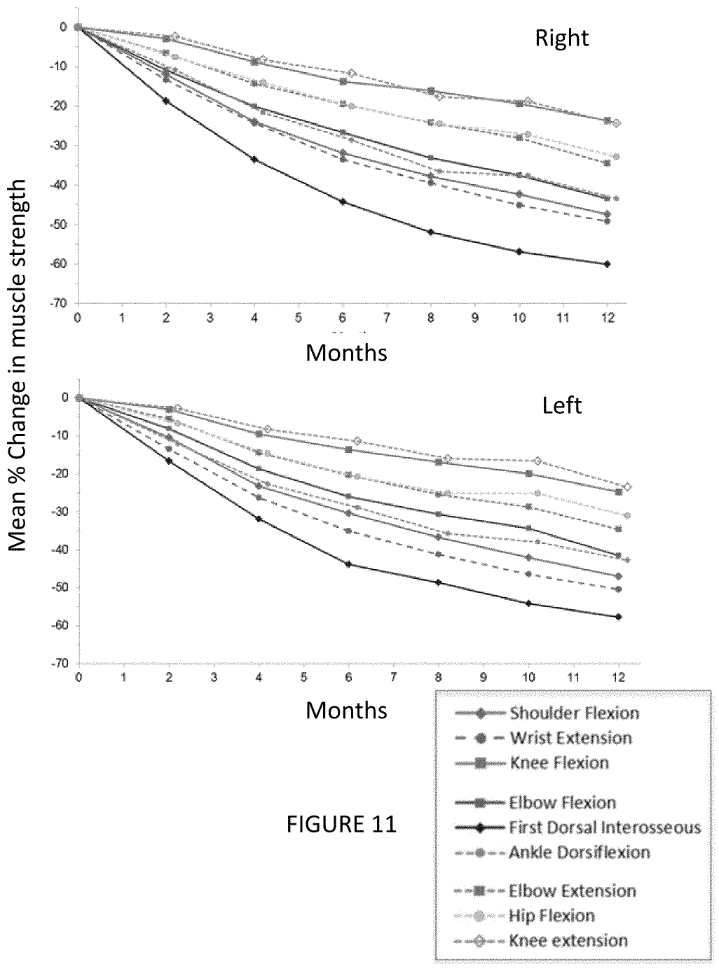

Muscle strength loss was measured over time for 18 muscle pairs in the EMPOWER trial using the microFET2 Hand Held Dynamometer (FIG. 11). Decline of strength was seen across all muscles tested and varied across muscle groups. Decline of strength was accentuated distally. This suggests that the more pronounced muscle measurements, and possibly more expeditious and/or prophetic of disease progression, are those of more distal muscles.

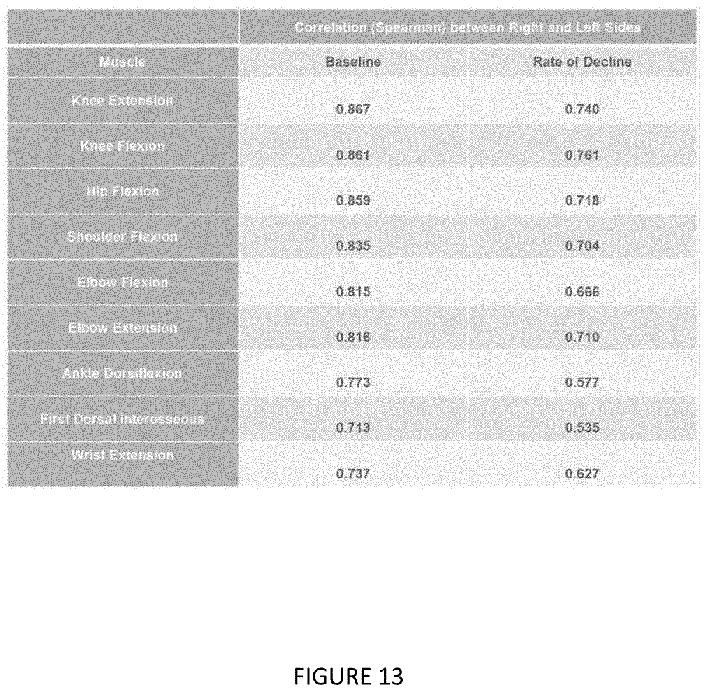

The correlation (Spearman's coefficient) of baseline strength and rate of strength decline between right side and left side muscles of subjects in the EMPOWER study was determined (FIG. 13). Strength at baseline and strength decline are consistent between right and left side muscles of a given pair for all muscles evaluated. This suggests that monitoring the strength and/or strength decline of one muscle of a right/left pair may be sufficient for evaluating that pair's muscle strength.

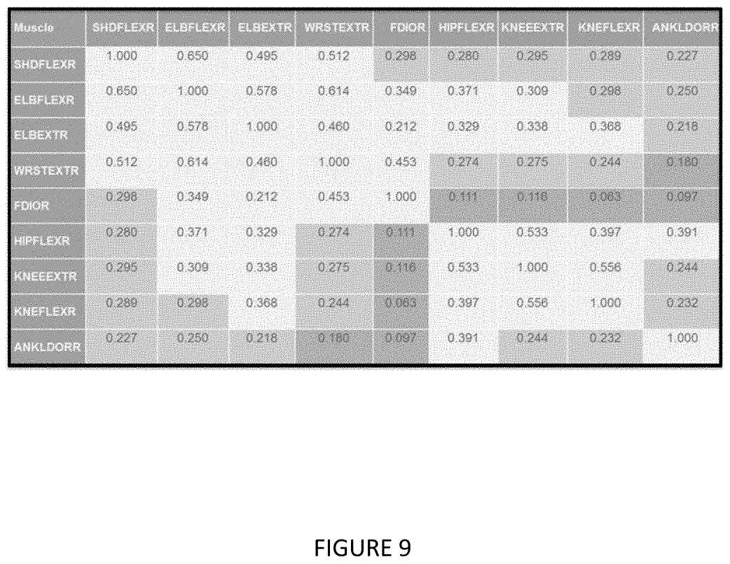

The correlation (Spearman's coefficient) of muscle strength loss between right side muscles in subjects in the EMPOWER study was determined (FIG. 9). Within each limb, the greatest correlation was found with neighboring muscles. Much less correlation was found between arm and leg muscles. This suggests that it may not be necessary to extensively gather data on every muscle in a limb in future studies.

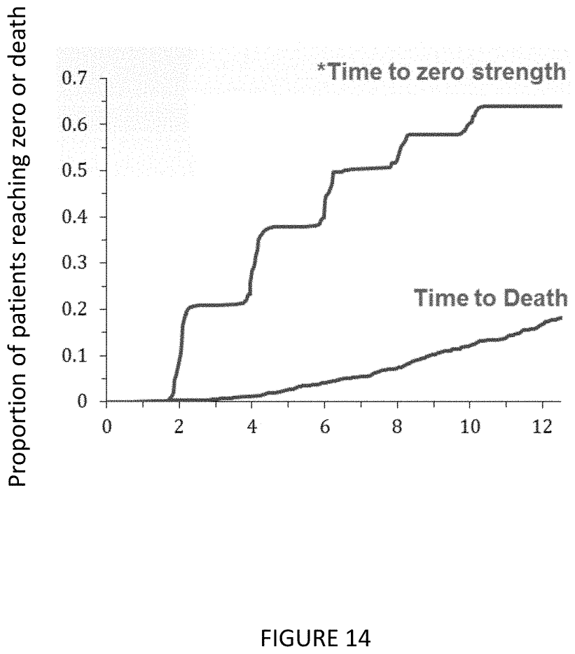

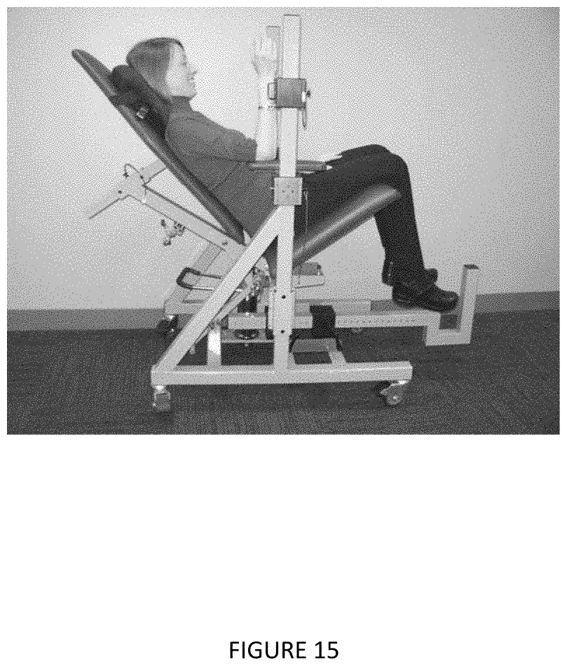

To create a new endpoint so that patients who had FDIO or ANKLDOR at zero strength at study entry could provide informative outcomes, ELBEXT and KNEEEXT were added to the definition. The modified new endpoint for zero strength is thus defined as the time of first observed zero force in any of the four muscles (FDIO, ANKLDOR, ELBEXT, and KNEEEXT) that is not zero at baseline (and subsequently confirmed). FIG. 14 plots the proportion of patients reaching this new zero strength endpoint in comparison to time to death (Kaplan-Meier estimates of survival). The time to zero strength, under the newly defined endpoint, correlates with time to death but is far more sensitive in terms of the proportion of patients who develop changes in the metric.

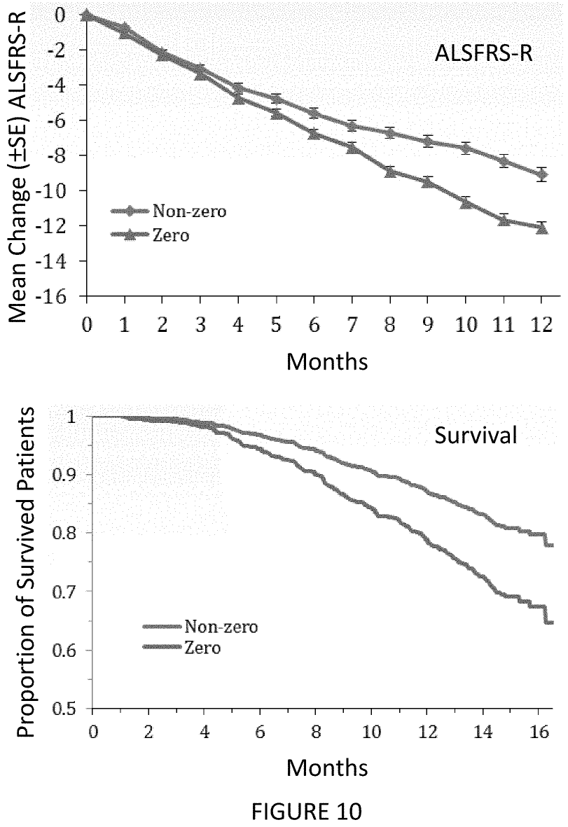

The population of patients experiencing the new zero strength endpoint was compared to the population not experiencing the new zero point endpoint with regards to other ALS endpoint metrics (e.g., ALSFRS-R and death/survival). The top graph of FIG. 10 shows that subjects experiencing zero strength saw a decreased ALSFRS-R and increased rate of decline of ALSFRS-R over a 12-month period relative to subjects not experiencing zero strength. The bottom graph of FIG. 10 shows that survival of subjects experiencing zero strength over a 12-month period decreased faster than the survival of subjects not experiencing zero strength. This suggests that the new zero strength endpoint represents a meaningful distinction defined by measurable, distinct functional and clinical outcomes in patients.

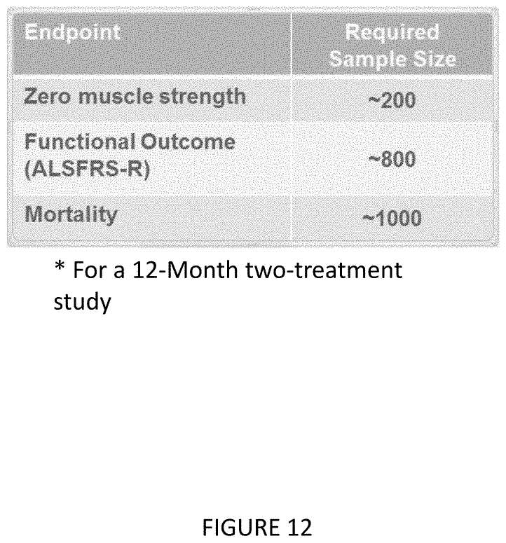

The new zero strength endpoint can enhance future ALS clinical studies. FIG. 12 shows the projected sample size requirements for a 12-month two-treatment study based on the zero strength endpoint, as well as ALSFRS-R and survival. The sample sizes were estimated to achieve 90% power to detect a 50% reduction in hazard of reaching muscle score zero, a mean treatment difference of 2 points for ALSFRS-R, and an 80% reduction in hazard of deaths at a level of 0.05 (2-sided). Under those requirements, approximately 200 subjects would be required to detect the change in reaching muscle strength zero, approximately 800 subjects would be required to detect a change in functional outcome via ALSFRS-R, and approximately 1000 subjects would be required to detect a reduction in deaths. This suggests the new zero strength endpoint may be useful in future ALS clinical studies, providing enhanced efficiency and predictive value over or alongside other metrics.

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of evaluating a subject having a disease associated with loss of muscle function comprising:
measuring a value for Muscle Function or for a Zero Muscle Function Factor (ZMF Factor) for a muscle of the subject; and
determining the time elapsed between a preselected event and the time at which Zero Function Value is reached (T_{ZFV}),
thereby evaluating the subject having a disease associated with loss of muscle function,
wherein the value for Muscle Function or for a ZMF Factor for a muscle of the subject is measured by a device, and
wherein the muscle is a Sentinel Muscle.

2. The method of claim 1, further comprising, comparing the value for T_{ZFV} with a reference value, and, optionally, responsive to the value for T_{ZFV}, classifying the subject.

3. (canceled)

4. The method of claim 1, further comprising determining if the value for ZMF Factor or Muscle Function, is a Zero Function Value; or comparing the value for ZMF Factor or Muscle Function with a reference value to determine if the muscle has reached Zero Function.

5. (canceled)

6. The method of claim 1, comprising providing a value for Muscle Function or for a Zero Muscle Function Factor (ZMF Factor):
) once, twice, three times, four times, five times, six times, seven times, eight times, nine times, or ten or more times; or
ii) once a month for at least 1, 2, 3, 4, 5, 6, 12, 18, or 24 or more months.

7. (canceled)

8. The method of claim 1, further comprising, responsive to the value for ZMF Factor or Muscle Function, classifying the subject.

9-11. (canceled)

12. The method of claim 1, wherein the disease associated with loss of muscle function is a motor neuron disease, neuromuscular disorder, or a myopathy.

13. The method of claim 1, wherein the disease associated with loss of muscle function is one or more selected from: amyotrophic lateral sclerosis (ALS), spinal muscular atrophy (SMA), spinobulbar muscular atrophy (SBMA), polymyositis, inclusion body myositis, a motor neuropathy, and distal hereditary motor neuropathy.

14.-15. (canceled)

16. The method of claim 1, wherein the value for Muscle Function or for a ZMF Factor for a muscle of the subject is measured by or expressed as one or more of:
a) the ability to exert force;
b) peak force;
c) the ability to maintain force;
d) an electrophysiological parameter, selected from: neuron conductivity, neuromuscular junction transmission, or ability of an axon to elicit a contraction of a muscle;
e) the ability to assume an anti-gravity position;
f) range of motion;
g) speed attainable; or
h) acceleration.

17. The method of claim 1, wherein evaluating the subject comprises one or more of:
evaluating disease progression;
evaluating for Zero Function Value for a muscle;
determining if a muscle in a subject has reached Zero Function;
determining prognosis;
predicting the outcome of the disease, in the subject;
predicting the survival of the subject;
evaluating the effectiveness of a treatment;
determining a preferred treatment regimen;
classifying the subject as having reached Zero Function for a muscle;
classifying the subject as having not reached Zero Function for a muscle;
comparing the Muscle Function, ZMF Factor, or T_{ZFV} slope to a ALSFRS-R slope;
comparing the Muscle Function, ZMF Factor, or T_{ZFV} slope to a survival slope; or
identifying a pattern of disease progression.

18. The method of claim 1, wherein evaluating the subject comprises, responsive to the value for Muscle Function, ZMF Factor, or T_{ZFV}, classifying, selecting, modifying prognosis or treatment or making a prediction about, the subject.

19. The method of claim 18, wherein evaluating, classifying, selecting, modifying prognosis or treatment or making a prediction about, the subject comprises one, two, or more of:
predicting the outcome of the disease in the subject;
predicting the survival of the subject;
evaluating the effectiveness of a treatment;
determining a preferred treatment regimen;
classifying the subject as having reached Zero Function for a muscle;
classifying the subject as having not reached Zero Function for a muscle;
comparing Muscle Function, ZMF Factor, or T_{ZFV} slope, to the ALSFRS-R slope;
comparing Muscle Function, ZMF Factor, or T_{ZFV} slope to the survival slope; or
identifying unusual patterns of disease progression.

20.-23. (canceled)

24. The method of claim 1, further comprising:
i) continuing a preexisting treatment under a lower or higher dosage or more or less frequent administrations; or
ii) continuing a preexisting treatment, and initiating a second treatment; or
iii) discontinuing a preexisting treatment, and initiating a second treatment.

25.-26. (canceled)

27. The method of claim 1, further comprising responsive to the evaluation, classifying or selecting the subject.

28. The method of claim 17, wherein the predicting or classifying is performed within a preselected time selected from any of the following:
initial diagnosis of the disease associated with muscle function loss;
initial administration of a treatment for the disease associated with muscle function loss;
initiation into clinical trial;
completion of a clinical trial;
modification of treatment for the disease associated with muscle function loss;
abandonment or conclusion of a treatment for the disease associated with muscle function loss;
failure to respond to a treatment for the disease associated with muscle function loss;
presentation with a preselected symptom, or disease stage;
prior to presentation of a preselected symptom;
1 day, 10 days, 20 days, 30 days, 60 days, 90 days, 120 days, 150 days, 180 days, 210 days, 240 days, 270 days, 300 days, 330 days, 365 days, 14 months, 16 months, 18 months, 20 months, 22 months, 24 months, 30 months, or 36 months; or
less than 48 months, less than 42 months, less than 36 months, less than 30 months, less than 24 months, less than 16 months, or less than 12 months.

29.-31. (canceled)

32. The method of claim 1, wherein:
a) responsive to the determination that the subject has not reached Zero Function, classifying the subject as being in need of subsequent evaluation; or
b) responsive to a value for Muscle Function or ZMF Factor based determination that the subject has reached Zero Function, classifying the subject as having Zero Function; or
c) responsive to a value for Muscle Function or ZMF Factor based determination that the subject has not reached Zero Function, classifying the subject as being in need of a subsequent evaluation for Zero Function.

33.-45. (canceled)

46. The method of claim 1, further comprising measuring a value for ZMF Factor or Muscle Function in one or more muscles selected from the:
left or right shoulder flexor (SHDFLEX);
left or right elbow flexion (ELBFLEX);
left or right first interosseous contraction (FDIO)
left or right ankle dorsiflexion (ANKLDOR);
left or right knee extensor (KNEEEXT);
left or right wrist extensor (WRSTEXT);
left or right elbow extensor (ELBEXT);
left or right knee flexor (KNEEFLEX); and
left or right hip flexor (HIPFLEX).

47.-50. (canceled)

51. The method of claim 1, wherein two, three, four, five, six, seven, eight, nine, ten, or more, muscles are evaluated.

52.-53. (canceled)

54. The method of claim 1, wherein a plurality of muscles are evaluated and each muscle of the plurality is one of a contralateral pair.

55. The method of claim 1, wherein the muscle is one which reaches, or is expected to reach, Zero Function at or after:
i) a preselected time; or
ii) 1, 2, 4 or 6 months from a preselected point selected from: after diagnosis of the disease, after initiation of a treatment, or after completion of a treatment; or
iii) a preselected Sentinel Muscle reaches, or is expected to reach, Zero Function; or
iv) one or more or all of, the L-FDIO, the R-FDIO, the L-ANKLDOR, and the R-ANKLDOR reaches, or is expected to reach, Zero Function.

56.-59. (canceled)

60. The method of claim 1, wherein the muscle is one which reaches, or is expected to reach, Zero Function prior to:
i) a preselected time; or
ii) a preselected Sentinel Muscle reaches, or is expected to reach, Zero Function; or
iii) one or more or all of, the L-FDIO, the R-FDIO, the L-ANKLDOR, and the R-ANKLDOR, reaches or is expected to reach Zero Function.

61.-85. (canceled)

86. The method of claim 1, wherein the ZMF Factor comprises a value for muscle function for:
(i) one FDIO but not the contralateral FDIO and one ANKLDOR but not the contralateral ANKLDOR; or
(ii) a FDIO, an ANKLDOR, an ELBEXT, and a KNEEEXT, optionally wherein the ZMF Factor comprises a value for muscle function for one FDIO, ANKLDOR, ELBEXT, or KNEEEXT, but not the contralateral FDIO, ANKLDOR, ELBEXT, or KNEEEXT.

87.-107. (canceled)

108. The method of claim 1, comprising measuring a value for muscle function for:
(i) one FDIO but not the contralateral FDIO and one ANKLDOR but not the contralateral ANKLDOR; or
(ii) a FDIO, an ANKLDOR, an ELBEXT, and a KNEEEXT, optionally wherein the ZMF Factor comprises a value for muscle function for one FDIO, ANKLDOR, ELBEXT, or KNEEEXT, but not the contralateral FDIO, ANKLDOR, ELBEXT, or KNEEEXT.

109.-113. (canceled)

114. The method of claim 1, wherein Muscle Function is determined by measurement of:
i) mechanical force; or
ii) electrophysiological signals.

115.-119. (canceled)

120. The method of claim 1, comprising correlating the state or progression of the subject's disease, expressed as a function of Muscle Function or for a Zero Muscle Function Factor (ZMF Factor), with one, two, three, or more of genotype, phenotype, family history, disease history, an environmental factor, mobility, or a selected activity.

121.-124. (canceled)

125. The method of claim 120, comprising, responsive to a correlation, classifying the subject, selecting a course of treatment, or administering a course of treatment to the subject.

126. A method of evaluating a subject having a motor neuron disease or related neuromuscular disorder, comprising:
a) measuring a value for Muscle Function or for a ZMF Factor for a muscle of the subject;
b) comparing the Muscle Function or ZMF Factor to a Zero Function Value to determine if the muscle has reached or has not reached zero-function;
thereby, responsive to the ZMF Factor or Muscle Function, evaluating the subject.

127. A method of evaluating or treating a subject having a disease associated with loss of muscle function, comprising:
determining a value from an evaluation made by the method of claim 1;
thereby evaluating or treating the subject.

128. The method of claim 127 comprising:
a) responsive to the value, selecting a treatment for the subject; or
b) acquiring from another entity a selection of treatment for the subject, the selection having been made responsive to the value; or
c) administering a treatment to the subject.

129. (canceled)

130. A method of evaluating a subject having a disease associated with loss of muscle function, comprising:
measuring, a value for Muscle Function or for a Zero Muscle Function Factor (ZMF Factor) for a muscle of the subject;
determining the time elapsed between a preselected event and the time at which Zero Function Value is reached (T_{ZFV}); and
responsive to the value for Muscle Function, ZMF Factor, or T_{ZFV}, classifying, selecting, modifying prognosis or treatment or making a prediction about, the subject,
wherein the value for Muscle Function or for a ZMF Factor for a muscle of the subject is measured by a device, and wherein the muscle is a Sentinel Muscle,
thereby evaluating the subject having a disease associated with loss of muscle function.
